SEQUENCE LISTING (137 sequences)

SEQ ID NO: 1 - protein, 28 aa, Artificial Sequence
  Synthetic
EKPYKCPECGKSFSASAALVAHQRTHTG

SEQ ID NO: 2 - protein, 56 aa, Artificial Sequence
  Synthetic
EKPYKCPECGKSFSASAALVAHQRTHTGEKPYKCPECGKSFSASAALVAHQRTHTG

SEQ ID NO: 3 - protein, 84 aa, Artificial Sequence
  Synthetic
EKPYKCPECGKSFSASAALVAHQRTHTGEKPYKCPECGKSFSASAALVAHQRTHTGEKPYKCPECGKSFSASAALVAHQRTHTG

SEQ ID NO: 4 - protein, 112 aa, Artificial Sequence
  Synthetic
EKPYKCPECGKSFSASAALVAHQRTHTGEKPYKCPECGKSFSASAALVAHQRTHTGEKPYKCPECGKSFSASAALVAHQRTHTGEKPYKCPECGKSFSASAALVAHQRTHTG

SEQ ID NO: 5 - protein, 38 aa, Artificial Sequence
  Synthetic
RIPLSCTICRKRKVKCDKLRPHCQQCTKTGVAHLCHYM

SEQ ID NO: 6 - protein, 17 aa, Artificial Sequence
  Synthetic
TCYNCGQTGHLSRECPS

SEQ ID NO: 7 - protein, 52 aa, Artificial Sequence
  Synthetic
RRCNCMATRHPLFEVAPNCLNCGKIICEKEGLQPCSYCGQELLSPKDKQEII

SEQ ID NO: 8 - protein, 425 aa, Artificial Sequence
  Synthetic
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEF

SEQ ID NO: 9 - protein, 871 aa, Artificial Sequence
  Synthetic
YKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 10 - protein, 1296 aa, Artificial Sequence [sequence omitted]

SEQ ID NO: 11 - protein, 518 aa, Artificial Sequence
  Synthetic
EKPYKCPECGKSFSASAALVAHQRTHTGEKPYKCPECGKSFSASAALVAHQRTHTGEKPYKCPECGKSFSASAALVAHQRTHTGGGSGGSGGSMPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEF

SEQ ID NO: 12 - protein, 1389 aa, Artificial Sequence
  Synthetic
EKPYKCPECGKSFSASAALVAHQRTHTGEKPYKCPECGKSFSASAALVAHQRTHTGEKPYKCPECGKSFSASAALVAHQRTHTGGGSGGSGGSMPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 13 - protein, 517 aa, Artificial Sequence
  Synthetic
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFGGSGGSGGSEKPYKCPECGKSFSASAALVAHQRTHTGEKPYKCPECGKSFSASAALVAHQRTHTGEKPYKCPECGKSFSASAALVAHQRTHT

SEQ ID NO: 14 - protein, 1388 aa, Artificial Sequence
  Synthetic
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLGGSGGSGGSEKPYKCPECGKSFSASAALVAHQRTHTGEKPYKCPECGKSFSASAALVAHQRTHTGEKPYKCPECGKSFSASAALVAHQRTHT

SEQ ID NO: 15 - protein, 1482 aa, Artificial Sequence
  Synthetic
EKPYKCPECGKSFSASAALVAHQRTHTGEKPYKCPECGKSFSASAALVAHQRTHTGEKPYKCPECGKSFSASAALVAHQRTHTGGGSGGSGGSMPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLGGSGGSGGSEKPYKCPECGKSFSASAALVAHQRTHTGEKPYKCPECGKSFSASAALVAHQRTHTGEKPYKCPECGKSFSASAALVAHQRTHTG

SEQ ID NO: 16 - protein, 1317 aa, Artificial Sequence
  Synthetic
GRKKRRQRRRPQGGSGGSGGSMPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 17 - protein, 1326 aa, Artificial Sequence
  Synthetic
KETWWETWWTEWSQPKKKRKVGGSGGSGGSMPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 18 - protein, 12 aa, Artificial Sequence
  Synthetic
GRKKRRQRRRPQ

SEQ ID NO: 19 - protein, 21 aa, Artificial Sequence
  Synthetic
KETWWETWWTEWSQPKKKRKV

SEQ ID NO: 20 - protein, 16 aa, Artificial Sequence
  Synthetic
RKKRRRESRKKRRRES

SEQ ID NO: 21 - protein, 17 aa, Artificial Sequence
  Synthetic
GRPRESGKKRKRKRLKP

SEQ ID NO: 22 - protein, 15 aa, Artificial Sequence
  Synthetic
GKRKKKGKLGKKRDP

SEQ ID NO: 23 - protein, 17 aa, Artificial Sequence
  Synthetic
GKRKKKGKLGKKRPRSR

SEQ ID NO: 24 - protein, 18 aa, Artificial Sequence
  Synthetic
RKKRRRESRRARRSPRHL

SEQ ID NO: 25 - protein, 19 aa, Artificial Sequence
  Synthetic
SRRARRSPRESGKKRKRKR

SEQ ID NO: 26 - protein, 19 aa, Artificial Sequence
  Synthetic
VKRGLKLRHVRPRVTRMDV

SEQ ID NO: 27 - protein, 14 aa, Artificial Sequence
  Synthetic
SRRARRSPRHLGSG

SEQ ID NO: 28 - protein, 16 aa, Artificial Sequence
  Synthetic
LRRERQSRLRRERQSR

SEQ ID NO: 29 - protein, 22 aa, Artificial Sequence
  Synthetic
GAYDLRRRERQSRLRRRERQSR

SEQ ID NO: 30 - protein, 9 aa, Artificial Sequence
  Synthetic
RKKRRQRRR

SEQ ID NO: 31 - protein, 15 aa, Artificial Sequence
  Synthetic
RRRRNRTRRNRRRVR

SEQ ID NO: 32 - protein, 17 aa, Artificial Sequence
  Synthetic
TRQARRNRRRRWRERQR

SEQ ID NO: 33 - protein, 13 aa, Artificial Sequence
  Synthetic
TRRQRTRRARRNR

SEQ ID NO: 34 - protein, 19 aa, Artificial Sequence
  Synthetic
KMTRAQRRAAARRNRWTAR

SEQ ID NO: 35 - protein, 17 aa, Artificial Sequence
  Synthetic
NAKTRRHERRRKLAIER

SEQ ID NO: 36 - protein, 22 aa, Artificial Sequence
  Synthetic
MDAQTRRRERRAEKQAQWKAAN

SEQ ID NO: 37 - protein, 18 aa, Artificial Sequence
  Synthetic
TAKTRYKARRAELIAERR

SEQ ID NO: 38 - protein, 16 aa, Artificial Sequence
  Synthetic
TRRNKRNRIQEQLNRK

SEQ ID NO: 39 - protein, 32 aa, Artificial Sequence
  Synthetic
PRRRRSSSRPVRRRRRPRVSRRRRRRGGRRRR

SEQ ID NO: 40 - protein, 28 aa, Artificial Sequence
  Synthetic
RIKAERKRMRNRIAASKSRKRKLERIAR

SEQ ID NO: 41 - protein, 26 aa, Artificial Sequence
  Synthetic
KRRIRRERNKMAAAKSRNRRRELTDT

SEQ ID NO: 42 - protein, 22 aa, Artificial Sequence
  Synthetic
KRARNTEAARRSRARKLQRMKQ

SEQ ID NO: 43 - protein, 16 aa, Artificial Sequence
  Synthetic
RQIKIWFQNRRMKWKK

SEQ ID NO: 44 - protein, 16 aa, Artificial Sequence
  Synthetic
RVIRVWFQNKRCKDKK

SEQ ID NO: 45 - protein, 60 aa, Artificial Sequence
  Synthetic
SKRTRQTYTRYQTLELEKEFHFNRYITRRRRIDIANALSLSERQIKIWFQNRRMKSKKDR

SEQ ID NO: 46 - protein, 16 aa, Artificial Sequence
  Synthetic
SQIKIWFQNKRAKIKK

SEQ ID NO: 47 - protein, 16 aa, Artificial Sequence
  Synthetic
RQVTIWFQNRRVKEKK

SEQ ID NO: 48 - protein, 16 aa, Artificial Sequence
  Synthetic
KQINNWFINQRKRHWK

SEQ ID NO: 49 - protein, 16 aa, Artificial Sequence
  Synthetic
RHIKIWFQNRRMKWKK

SEQ ID NO: 50 - protein, 23 aa, Artificial Sequence
  Synthetic
MGLGLHLLVLAAALQGAKKKRKV

SEQ ID NO: 51 - protein, 30 aa, Artificial Sequence
  Synthetic
MVKSKIGSWILVLFVAMWSDVGLCKKRPKP

SEQ ID NO: 52 - protein, 28 aa, Artificial Sequence
  Synthetic
MANLGYWLLALFVTMWTDVGLCKKRPKP

SEQ ID NO: 53 - protein, 21 aa, Artificial Sequence
  Synthetic
AAVLLPVLLAAPVQRKRQKLP

SEQ ID NO: 54 - protein, 12 aa, Artificial Sequence
  Synthetic
AAVLLPVLLAAP

SEQ ID NO: 55 - protein, 24 aa, Artificial Sequence
  Synthetic
RRIRPRPPRLPRPRPRPLPFPRPG

SEQ ID NO: 56 - protein, 20 aa, Artificial Sequence
  Synthetic
VDKGSYLPRPTPPRPIYNRN

SEQ ID NO: 57 - protein, 22 aa, Artificial Sequence
  Synthetic
KCFQWQRNMRKVRGPPVSCIKR

SEQ ID NO: 58 - protein, 21 aa, Artificial Sequence
  Synthetic
TRSSRAGLQWPVGRVHRLLRK

SEQ ID NO: 59 - protein, 26 aa, Artificial Sequence
  Synthetic
GIGAVLKVLTTGLPALISWIKRKRQQ

SEQ ID NO: 60 - protein, 23 aa, Artificial Sequence
  Synthetic
GIGKWLHSAKKFGKAFVGEIMNS

SEQ ID NO: 61 - protein, 38 aa, Artificial Sequence
  Synthetic
LLGDFFRKSKEKIGKEFKRIVQRIKDFLRNLVPRTESC

SEQ ID NO: 62 - protein, 18 aa, Artificial Sequence
  Synthetic
RGGRLSYSRRRFSTSTGR

SEQ ID NO: 63 - protein, 14 aa, Artificial Sequence
  Synthetic
YKQCHKKGGKKGSG

SEQ ID NO: 64 - protein, 20 aa, Artificial Sequence
  Synthetic
ALWKTLLKKVLKAPKKKRKV

SEQ ID NO: 65 - protein, 20 aa, Artificial Sequence
  Synthetic
HARIKPTFRRLKWKYKGKFW

SEQ ID NO: 66 - protein, 22 aa, Artificial Sequence
  Synthetic
TKRRITPKDVIDVRSVTTEINT

SEQ ID NO: 67 - protein, 27 aa, Artificial Sequence
  Synthetic
RQGAARVTSWLGRQLRIAGKRLEGRSK

SEQ ID NO: 68 - protein, 34 aa, Artificial Sequence
  Synthetic
NAATATRGRSAASRPTQRPRAPARSASRPRRPVQ

SEQ ID NO: 69 - protein, 16 aa, Artificial Sequence
  Synthetic
RHSRIGIIQQRRTRNG

SEQ ID NO: 70 - protein, 29 aa, Artificial Sequence
  Synthetic
KLIKGRTPIKFGKADCDRPPKHSQNGMGK

SEQ ID NO: 71 - protein, 12 aa, Artificial Sequence
  Synthetic
PLSSIFSRIGDP

SEQ ID NO: 72 - protein, 26 aa, Artificial Sequence
  Synthetic
DPKGDPKGVTVTVTVTVTGKGDPKPD

SEQ ID NO: 73 - protein, 10 aa, Artificial Sequence
  Synthetic
RRIPNRRPRR

SEQ ID NO: 74 - protein, 5 aa, Artificial Sequence
  Synthetic
RLRWR

SEQ ID NO: 75 - protein, 22 aa, Artificial Sequence
  Synthetic
MVRRFLVTLRIRRACGPPRVRV

SEQ ID NO: 76 - protein, 22 aa, Artificial Sequence
  Synthetic
MVTVLFRRLRIRRACGPPRVRV

SEQ ID NO: 77 - protein, 18 aa, Artificial Sequence
  Synthetic
LLIILRRRIRKQAHAHSK

SEQ ID NO: 78 - protein, 18 aa, Artificial Sequence
  Synthetic
LSTAADMQGVVTDGMASG

SEQ ID NO: 79 - protein, 28 aa, Artificial Sequence
  Synthetic
LSTAADMQGVVTDGMASGLDKDYLKPDD

SEQ ID NO: 80 - protein, 15 aa, Artificial Sequence
  Synthetic
KFHTFPQTAIGVGAP

SEQ ID NO: 81 - protein, 5 aa, Artificial Sequence
  Synthetic
VPTLK

SEQ ID NO: 82 - protein, 5 aa, Artificial Sequence
  Synthetic
PMLKE

SEQ ID NO: 83 - protein, 5 aa, Artificial Sequence
  Synthetic
VPALR

SEQ ID NO: 84 - protein, 5 aa, Artificial Sequence
  Synthetic
VSALK

SEQ ID NO: 85 - protein, 5 aa, Artificial Sequence
  Synthetic
IPALK

SEQ ID NO: 86 - protein, 6 aa, Artificial Sequence
  Synthetic
PFVYLI

SEQ ID NO: 87 - protein, 10 aa, Artificial Sequence
  Synthetic
PIEVCMYREP

SEQ ID NO: 88 - protein, 8 aa, Artificial Sequence
  Synthetic
RRRRRRRR

SEQ ID NO: 89 - protein, 9 aa, Artificial Sequence
  Synthetic
RRRRRRRRR

SEQ ID NO: 90 - protein, 10 aa, Artificial Sequence
  Synthetic
RRRRRRRRRR

SEQ ID NO: 91 - protein, 12 aa, Artificial Sequence
  Synthetic
RRRRRRRRRRRR

SEQ ID NO: 92 - protein, 20 aa, Artificial Sequence
  Synthetic
KETWWETWWTEWSQPKKRKV

SEQ ID NO: 93 - protein, 27 aa, Artificial Sequence
  Synthetic
GLAFLGFLGAAGSTMGAWSQPKKKRKV

SEQ ID NO: 94 - protein, 27 aa, Artificial Sequence
  Synthetic
GWTLNSAGYLLGKINLKALAALAKKIL

SEQ ID NO: 95 - protein, 23 aa, Artificial Sequence
  Synthetic
AGYLLGHINLHHLAHLAIBHHIL

SEQ ID NO: 96 - protein, 17 aa, Artificial Sequence
  Synthetic
KLALKALKALKAALKLA

SEQ ID NO: 97 - protein, 9 aa, Artificial Sequence
  Synthetic
RRWWRRWRR

SEQ ID NO: 98 - protein, 20 aa, Artificial Sequence
  Synthetic
GLWRALWRLLRSLWRLLWRA

SEQ ID NO: 99 - protein, 23 aa, Artificial Sequence
  Synthetic
LIRLWSHLIHIWFQNRRLKWKKK

SEQ ID NO: 100 - protein, 30 aa, Artificial Sequence
  Synthetic
WEAALAEALAEALAEHLAEALAEALEALAA

SEQ ID NO: 101 - protein, 18 aa, Artificial Sequence
  Synthetic
LKTLTETLKELTKTLTEL

SEQ ID NO: 102 - protein, 18 aa, Artificial Sequence
  Synthetic
QLALQLALQALQAALQLA

SEQ ID NO: 103 - protein, 9 aa, Artificial Sequence
  Synthetic
PPRPPRPPR

SEQ ID NO: 104 - protein, 12 aa, Artificial Sequence
  Synthetic
PPRPPRPPRPPR

SEQ ID NO: 105 - protein, 15 aa, Artificial Sequence
  Synthetic
PPRPPRPPRPPRPPR

SEQ ID NO: 106 - protein, 18 aa, Artificial Sequence
  Synthetic
PPRPPRPPRPPRPPRPPR

SEQ ID NO: 107 - protein, 9 aa, Artificial Sequence
  Synthetic
PRRPRRPRR

SEQ ID NO: 108 - protein, 12 aa, Artificial Sequence
  Synthetic
PRRPRRPRRPRR

SEQ ID NO: 109 - protein, 15 aa, Artificial Sequence
  Synthetic
PRRPRRPRRPRRPRR

SEQ ID NO: 110 - protein, 18 aa, Artificial Sequence
  Synthetic
PRRPRRPRRPRRPRRPRR

SEQ ID NO: 111 - protein, 36 aa, Artificial Sequence
  Synthetic
GPSQPTYPGDDAPVRDLIRFYRDLQRYLNVVTRHRY

SEQ ID NO: 112 - protein, 36 aa, Artificial Sequence
  Synthetic
GPSQPTYPGDDAPVRDLIRFYRDLRRYLNVVTRHRY

SEQ ID NO: 113 - protein, 36 aa, Artificial Sequence
  Synthetic
GPSQPTYPGDDAPVRDLRRFYRDLRRYLNVVTRHRY

SEQ ID NO: 114 - protein, 5 aa, Artificial Sequence
  Synthetic
  MISC_FEATURE (2)..(4): Xaa can be any amino acid and residues 2-4 can repeat
GPXXP

SEQ ID NO: 115 - protein, 18 aa, Artificial Sequence
  Synthetic
VRLPPPVRLPPPVRLPPP

SEQ ID NO: 116 - protein, 18 aa, Artificial Sequence
  Synthetic
VELPPPVELPPPVELPPP

SEQ ID NO: 117 - protein, 15 aa, Artificial Sequence
  Synthetic
FKIYDKKVRTRVVKH

SEQ ID NO: 118 - protein, 18 aa, Artificial Sequence
  Synthetic
RASKRDGSWVKKLHRILE

SEQ ID NO: 119 - protein, 16 aa, Artificial Sequence
  Synthetic
KGTYKKKLMRIPLKGT

SEQ ID NO: 120 - protein, 19 aa, Artificial Sequence
  Synthetic
LYKKGPAKKGRPPLRGWFH

SEQ ID NO: 121 - protein, 17 aa, Artificial Sequence
  Synthetic
HSPIIPLGTRFVCHGVT

SEQ ID NO: 122 - protein, 16 aa, Artificial Sequence
  Synthetic
YTAIAWVKAFIRKLRK

SEQ ID NO: 123 - protein, 17 aa, Artificial Sequence
  Synthetic
IAWVKAFIRKLRKGPLG

SEQ ID NO: 124 - protein, 14 aa, Artificial Sequence
  Synthetic
RLSGMNEVLSFRWL

SEQ ID NO: 125 - protein, 15 aa, Artificial Sequence
  Synthetic
SDLWEMMMVSLACQY

SEQ ID NO: 126 - protein, 12 aa, Artificial Sequence
  Synthetic
VTWTPQAWFQWV

SEQ ID NO: 127 - protein, 13 aa, Artificial Sequence
  Synthetic
GSPWGLQHHPPRT

SEQ ID NO: 128 - protein, 13 aa, Artificial Sequence
  Synthetic
GPFHFYQFLFPPV

SEQ ID NO: 129 - protein, 12 aa, Artificial Sequence
  Synthetic
TSPLNIHNGQKL

SEQ ID NO: 130 - protein, 18 aa, Artificial Sequence
  Synthetic
RCGRASRCRVRWMRRRRI

SEQ ID NO: 131 - protein, 26 aa, Artificial Sequence
  Synthetic
PYSRPHVQLWYPNRESCRSLIRSLGP

SEQ ID NO: 132 - protein, 12 aa, Artificial Sequence
  Synthetic
PLILLRLLRGQF

SEQ ID NO: 133 - protein, 12 aa, Artificial Sequence
  Synthetic
PLIYLRLLRGQF

SEQ ID NO: 134 - protein, 15 aa, Artificial Sequence
  Synthetic
KLWMRWYSPTTRRYG

SEQ ID NO: 135 - protein, 12 aa, Artificial Sequence
  Synthetic
  MISC_FEATURE (2)..(2): Arg is D-Arg
TRIDQANQRATK

SEQ ID NO: 136 - protein, 16 aa, Artificial Sequence
  Synthetic
HTQSLDQGYNDDDDKA

SEQ ID NO: 137 - protein, 17 aa, Artificial Sequence
  Synthetic
HTQSLDQGGENLYFQGA

(19) United States
(12) Patent Application Publication
(10) Pub. No.: US 2024/0216487 A1
(43) Pub. Date: Jul. 4, 2024

(54) INTRAMUSCULAR COMPOSITIONS OF BOTULINUM NEUROTOXINS

(71) Applicant: SHANGHAITECH UNIVERSITY, Shanghai (CN)

(72) Inventors: Jia LIU, Shanghai (CN); Xuan WEI, Shanghai (CN); Lu LI, Shanghai (CN)

(21) Appl. No.: 18/557,232

(22) PCT Filed: Apr. 26, 2022
(86) PCT No.: PCT/CN2022/089382

§ 371 Date: Oct. 25, 2023

(30) Foreign Application Priority Data
Apr. 26, 2021 (WO) PCT/CN2021/089918

(51) Int. Cl. A61K 38/48; A61K 9/00; A61K 38/17

(52) U.S. Cl. CPC A61K 38/4893; A61K 9/0019; A61K 38/1709; C12Y 304/24069

(57) ABSTRACT

The present disclosure provides fusion proteins combining botulinum neurotoxins (BoNTs) and cell penetration peptides (CPP) which are suitable for intramuscular administration. These fusion proteins have exceptional cellular uptake ability, potent therapeutic efficacy, and considerably increased therapeutic index when compared to the BoNT protein alone or the commercial product BOTOX® onabotulinumtoxinA. Also provided are BONT fusion proteins that are not cleaved to form two-chain polypeptides but yet retaining strong enzymatic activities. Such single-chain active BONT fusion proteins can be prepared from insect cells.

The present invention claims the priority of the PCT/CN2021/089918, filed on Apr. 26, 2021, the contents of which are incorporated herein by its entirety.

### BACKGROUND

Botulinum neurotoxins (BoNTs) are neurotoxic proteins produced by Clostridium botulinum and related bacterial species. Type A BoNT (BONTA) and its derivatives are a widely used aesthetic and therapeutic agents for treating neuromuscular disorders. BONTA is composed of a 100 kDa heavy chain (HC) and a 50 kDa light chain (LC). BONTA-HC binds to the SV2 receptor on motor nerve terminals which mediates the cellular uptake of BONTA. BONTA-LC specifically cleaves the 25-kD synaptosomal nerve-associated protein (SNAP-25) that is in charge of the docking and fusion of cellular vesicles.

Depending on complexed accessory proteins and formulated excipients, marketed BONTA products have therapeutic indexes ranging from 5 to 15 for intramuscular injection, as defined by the ratio of half maximum lethal dose (IMLD₅₀) and half maximum effective dose (IMED₅₀). The constantly expanding medical indications of BoNTA have led to an increasing number of serious and long-term adverse effects, underscoring the importance of improving the therapeutic index of BoNTA. Previous studies have shown that engineering of the LC domain can improve the therapeutic index of BONTA by up to 2 folds.

### SUMMARY OF THE INVENTION

The present disclosure provides fusion proteins combining botulinum neurotoxins (BoNTs) and cell penetration peptides (CPP) which are suitable for intramuscular administration. These CPP-BONT fusions have exceptional cellular uptake ability, potent therapeutic efficacy, and considerably increased therapeutic index when compared to the BoNT protein alone or the commercial product BOTOX® onabotulinumtoxinA. Also provided are BoNT fusion proteins that are not cleaved to form two-chain polypeptides but yet retaining strong enzymatic activities, which can be prepared from insect cells.

The present disclosure provides, in one embodiment, a method of delivering a botulinum toxin (BoNT) to a mammal, comprising intramuscularly administering a pharmaceutical composition comprising a polypeptide comprising a BoNT fused to a cell penetrating peptide (CPP), wherein the BoNT comprises a light chain and a heavy chain.

Non-limiting examples of CPP include a zinc finger peptide (ZFP, EKPYKCPECGKSFSASAALVAHQRTHTG, SEQ ID NO: 1), TAT (GRKKRRQRRRPQ, SEQ ID NO: 18) and Pep-1 (N-acetyl-KETWWETWWTEWSQPKKKRKV-OH, SEQ ID NO: 19), and those provided in Table 3.

The CPP may be fused to the N-terminus of the light chain of the BoNT, to the C-terminus of the heavy chain, or both, without limitation.

In some embodiments, at least 50%, preferably at least 75%, 80%, 85%, 90% or 95%, or all, of the BoNT in the composition are single-chain, i.e., the light chain and the heavy chain are on the same peptide chain.

In some embodiments, at least 50%, preferably at least 75%, 80%, 85%, 90% or 95% or all, of the BoNT in the composition are expressed from insect cells. The insect cells may be Spodoptera frugiperda cells or Trichoplusia ni cells, without limitation.

The intramuscular administration may be into any muscle in the mammal's body, such as under a skin or a mucous membrane of an eye, or at the car, nose, mouth, lip, urethral opening, anus, or tongue.

In some embodiments, the mammal is in need of treatment of facial wrinkle, dystonias, sparsticity, hemifacial spasm, hyperhidrosis, or hypersalivation. In some embodiments, the mammal is in need of muscle shaping.

Also provided is a pharmaceutical formulation comprising a pharmaceutically acceptable excipient and a polypeptide comprising a BoNT fused to a cell penetrating peptide (CPP), wherein the BoNT comprises a light chain and a heavy chain.

In some embodiments, the pharmaceutical formulation is lyophilized. In some embodiments, the pharmaceutical formulation is an injectable solution. In some embodiments, pharmaceutical formulation is formulated for intramuscular injection.

Non-limiting examples of CPP include a zinc finger peptide (ZFP, EKPYKCPECGKSFSASAALVAHQRTHTG, SEQ ID NO: 1), TAT (GRKKRRQRRRPQ. SEQ ID NO: 18) and Pep-1 (N-acetyl-KETWWETWWTEWSQPKKKRKV-OH, SEQ ID NO: 19), and those provided in Table 3.

The CPP may be fused to the N-terminus of the light chain of the BoNT, to the C-terminus of the heavy chain, or both, without limitation.

In some embodiments, at least 50%, preferably at least 75%, 80%, 85%, 90% or 95%, or all, of the BoNT in the composition are single-chain, i.e., the light chain and the heavy chain are on the same peptide chain.

In some embodiments, at least 50%, preferably at least 75%, 80%, 85%, 90% or 95% or all, of the BoNT in the composition are expressed from insect cells. The insect cells may be Spodoptera frugiperda cells or Trichoplusia ni cells, without limitation.

### BRIEF DESCRIPTION OF THE DRAWINGS

FIG. 1 illustrates the structures of various BONTA fusion proteins tested in the examples.

FIG. 2 shows the results of a SNAPtide assay of the in vitro activity of cell-penetrating BONTA proteins. Positive control is commercially available recombinant BONTA light chain (BONTA-LC, R&D Systems). Mock is the SNAPtide alone reaction.

FIG. 3 shows the results of a SNAPtide assay of the cell lysis of human dermal fibroblasts treated with cell-penetrating BoNTA proteins. Negative control is commercially available recombinant BONTA-LC (R&D Systems).

FIG. 4 presents representative images of the in vivo effects of cell-penetrating BONTA proteins.

FIG. 5 presents scatter plots of digit abduction of mice treated with cell-penetrating BONTA proteins. A, treated with 0.9% NaCl saline solution. B, injected with BOTOX. C, pre-treated with microneedle and then treated with BOTOX. D, pre-treated with microneedle and then treated with cell-penetrating BONTA-ZFP (Protein ID 6). E, treated with cell-penetrating BONTA-ZFP (Protein ID 6) without microneedle pre-treatment.

FIG. 6 is a schematic presentation showing the design of CPP-BONTA used in Example 3.

FIG. 7 shows the gel images of reduced SDS-PAGE gel with purified CPP-BONTA proteins.

FIG. 8 shows the testing results of solution stability of purified CPP-BONTA proteins. BONTA activity was determined using reporter peptide after incubation in aqueous solution at 4° C. for one month. Significant difference between BONTA and CPP-BONTA was determined using one-way ANOVA with Dunnett's multiple comparisons test.

FIG. 9 shows the quantification of the mean fluorescence intensity of BoNTA positive cells. Three biological replicates were performed and 1,000 cells were analyzed for each replicate.

FIG. 10 shows the results of in vitro activity of CPP-BONTA, as determined by the cleavage of a reporter peptide. Data are shown as mean±standard deviation (SD; n=8). Significant difference between WT and CPP-BONTA is determined using one-way ANOVA with Dunnett's multiple comparisons test.

FIG. 11 shows that CPP fusions improved cellular uptake mouse gastrocnemius muscle fibers, as determined by immunofluorescence experiments on sectioned tissues.

FIG. 12 shows quantification of print areas and stride length of BoNTA proteins before and after treatment. Gaits bearing synchronous rabbit-like hopping behaviors or elbow touch with the glass plate are defined as zero. Mice unable to complete the walk were removed from the analysis.

FIG. 13 shows the results of treadmill analysis of the in vivo potency of CPP-BONTA proteins

FIG. 14 shows titers of neutralizing antibodies induced by different BONTA proteins. It shows that TAT and bipartite ZFP fusions reduced the amount of neutralizing antibodies during repeated dosing.

FIG. 15 shows duration of muscle-weakening effects of CPP-BONTA after repeated dosing. TAT and bipartite ZFP fusions increased the duration of injected BONTA during repeated dosing. BoNTA proteins are injected at day 0, 30 and 47.

FIG. 16 shows the muscle shaping effects of BoNTA proteins on gastrocnemius muscle hypertrophy in C57BL/6 mouse. Images were taken when functional recovery of the gastrocnemius muscle is achieved. i.m., intramuscular injection.

FIG. 17 shows the influence of BoNTA proteins on gastrocnemius muscle hypertrophy in SD rats. Images are taken when functional recovery of the gastrocnemius muscle was achieved at 12 weeks after injection. Gastrocnemius muscles from the injected limb are highlighted by red boxes. Gastrocnemius muscles from the contralateral limb (mock) are highlighted by blue boxes.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acctylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively. That are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein the term “disulfide bond” includes the covalent bond formed between two sulfur atoms. The amino acid cysteine comprises a thiol group that can form a disulfide bond or bridge with a second thiol group.

As used herein, the terms “treat” or “treatment” refer to both therapeutic treatment and prophylactic or preventative measures, wherein the object is to prevent or slow down (lessen) an undesired physiological change or disorder, such as undesired wrinkles. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Intramuscular Administration of CPP-BONT Fusions

There is a need on the market for transdermal delivery of BONT proteins. Transdermal delivery of proteins is inherently difficult, in particular across the skin. Skin is constituted by two layers of cells, known as epidermis and dermis. Epidermis, the topmost layer of skin, is stratified squamous epithelium composing of basal and differentiated keratinocytes. Keratinocytes are the major cell types in epidermis. Keratinocytes in the basal stratum can proliferate through mitosis and undergo multiple cell differentiation stages to become anucleated cells. Anucleated or differentiated keratinocytes are highly organized tissue structure, secreting keratin proteins and lipids, which provide a protective barrier against invading substances such as pathogens.

Intramuscular delivery of the BoNT has been contemplated. There are still two major challenges. One is that the injected BONT still requires uptake by the target cell. The other is that intramuscular delivered BoNT tends to have low therapeutic index (safety margin), which causes safety concerns.

A first unexpected discovery of the present disclosure is that, while all tested CPP improved the cellular uptake of BoNT (FIG. 9) by cultured cells, the uptake was more significantly increased across the board when the BoNT proteins were intramuscularly injected (FIG. 11).

The second unexpected discovery is that the intramuscularly delivered CPP-BONT fusions had remarkably lower toxicity, especially when compared to BOTOX® onabotulinumtoxinA, a commercially available BONT product. For instance, TAT-BONTA was 7 times, Pep1-BONTA was 10 times, and ZFP3-BONTA-ZFP3 was a whopping 229 times safer than onabotulinumtoxinA (Table 6). Consequently, all of these CPP-BONT fusions exhibited considerably greater therapeutic indices than onabotulinumtoxinA (Table 6).

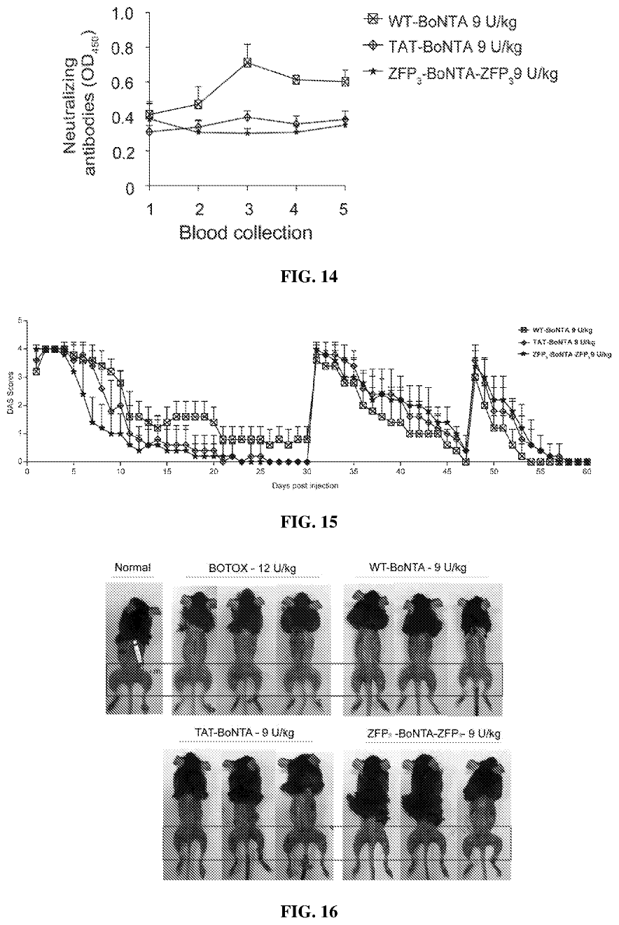

It is not entirely clear yet why these CPP-BONT fusions, when administered intramuscularly, had such excellent therapeutic indices. It is contemplated, however, that this is due to their greatly reduced immunogenicity (FIG. 14).

The third unexpected discovery is that all these tested CPP-BONT fusion proteins were actually single-chain proteins (FIG. 7). In nature, BoNT single-chain precursor proteins self-leave into two peptide chains, a BoNT light chain (LC) and a BoNT heavy chain (HC), connected via a disulfide bond. The single-chain precursor is known as relatively inactive. The potent activity of these single-chain fusion proteins, therefore, was entirely unexpected.

The lack of self-cleavage of the CPP-BONT proteins is contemplated to be because these proteins were expressed in insect cells (e.g., Spodoptera frugiperda cells or Trichoplusia ni cells).

The fourth unexpected discovery is that these CPP-BONT fusion proteins, when intramuscularly injected, exhibited significant muscle shaping (thinning) effects, while onabotulinumtoxinA only had modest results (FIG. 16). Therefore, the fusion proteins and compositions of the instant disclosure can have broader therapeutic functions than onabotulinumtoxinA, such as for reducing muscle mass, tightening or thinning muscle.

The fifth unexpected discovery is that, when administered intramuscularly, the fusion proteins exhibited significantly higher half-life after repeated dosing (FIG. 15). The increase duration, therefore, can help reduce the dosing frequency, adding to convenience of the described therapeutic methods.

In accordance with one embodiment of the present disclosure, therefore, provided is a method of delivering a botulinum toxin (BoNT) to a mammal. In some embodiments, the method entails intramuscularly administering a pharmaceutical composition comprising a polypeptide comprising a BoNT fused to a cell penetrating peptide (CPP), wherein the BoNT comprises a light chain and a heavy chain.

Cell-penetrating peptides (CPPs) are short (e.g., less than 200 amino acids in length) peptides that facilitate cellular intake and uptake of molecules ranging from nanosize particles to small chemical compounds to large fragments of DNA. CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake.

Transactivating transcriptional activator (TAT), from human immunodeficiency virus 1 (HIV-1), was the first CPP discovered. Additional CPPs were later discovered, spanning multiple categories and types. Non-limiting examples are provided in Table 3.

One example type of CPP is zinc finger proteins which are naturally occurring transcription factors and can be reprogrammed to recognize targeted genomic loci. Zinc finger nucleases-chimeric proteins containing an N-terminal ZFP domain and C-terminal Fok I endonuclease domain-have been shown to be intrinsically cell-permeable. Some of the ZFP include a Cys₂-His₂ ZFP domain. Cys₂-His₂ ZFPs consist of approximately 30 amino acids with a ββα configuration.

Another example is TAT (GRKKRRQRRRPQ, SEQ ID NO: 18, or simply RKKRRQRRR, SEQ ID NO: 30) is derived from the transactivator of transcription (TAT) of human immunodeficiency virus. Yet another example is Pep-1 (N-acetyl-KETWWETWWTEWSQPKKKRKV-OH, SEQ ID NO: 19) which is an amphipathic CPP—a first part is hydrophobic and contains several tryptophan residues (W) that can be involved in membrane destabilization processes, and a second part is cationic with lysine and arginine residues.

There are at least seven types of botulinum toxin, named type A-G. Type A and B are capable of causing disease in humans, and are also used commercially and medically. Types C-G are less common. Botulinum toxin types A and B are used in medicine to treat various muscle spasms and diseases characterized by overactive muscle. Each BONT serotype may also have subtypes. For instance, the following subtypes are known: BONT A1-A10, B1-B8, E1-E9, and F1-F7.

BONT proteins consist of a heavy chain and a light chain linked together by a single disulphide bond. They are synthesized as a relatively inactive single-chain polypeptide with a molecular mass of approximately 150 kDa, and are activated (to about 100-fold activity) when the polypeptide chain is proteolytically cleaved into the 100-kDa heavy chain and the 50-kDa light chain.

In some embodiments, the BoNT protein being administered or formulated is a single-chain protein, or at least a substantial portion of the composition being administered or formulated is single-chain, which is unexpected found to be active in the form of the CPP-BONT fusion protein. In some embodiments, at least 50%, 55%, 60%, 65%, 70%, 75%, 80%, 85%, 90%, 95%, 96%, 97%, 98%, 99%, 99.5%, 99.8%, 99.9% or all of the BoNT proteins are single-chain. In some embodiments, at least 50%, 55%, 60%, 65%, 70%, 75%, 80%, 85%, 90%, 95%, 96%, 97%, 98%, 99%, 99.5%, 99.8%, 99.9% or all of the BoNT proteins are single-chain and are not capable of cleaving itself into two chains.

In some embodiments, the single-chain BONT proteins are produced from insect cells. Example insect cells include Lepidoptera cells, Noctuidae cells, Spodoptera cells, and Spodoptera frugiperda cells. It is contemplated that the insect production system generates a BONT protein that is different from those produced from prokaryotic cells in terms of protein folding or post-translational modification. These proteins, therefore, are unable of self-cleavage.

In some embodiments, the BoNT included in the CPP-BONT fusion is a mutant BONT with one or more mutations that disable the cleavage. For instance, for BONTA, amino acid residues Lys438-Ala449 (residue numbers according to Protein ID 1) may be mutated to a different amino acid. In some embodiments, the mutation is a non-conservative mutation. Example mutations include, without limitation, HTQSLDQGYNDDDDKA (SEQ ID NO: 136) and HTQSLDQGGENLYFQGA (SEQ ID NO: 137).

In some embodiments, the CPP is located at the N-terminal side of the BoNT protein. In some embodiments, the CPP is located at the C-terminal side of the BoNT protein. In some embodiments, at least one CPP is located at the N-terminal side of the BoNT protein, and at least one CPP is located at the C-terminal side of the BoNT protein. In some embodiments, at either or both of the N-terminal and C-terminal sides, there are more than one CPP molecule.

The total size of the fusion (chimeric) polypeptide, in some embodiments, is not greater than 5000 amino acid residues, or alternatively not greater than 4000 amino acid residues, not greater than 3000 amino acid residues, not greater than 2000 amino acid residues, not greater than 1800 amino acid residues, not greater than 1600 amino acid residues, not greater than 1500 amino acid residues, not greater than 1400 amino acid residues, not greater than 1300 amino acid residues, not greater than 1200 amino acid residues, not greater than 1100 amino acid residues, not greater than 1000 amino acid residues, not greater than 900 amino acid residues, not greater than 800 amino acid residues, not greater than 700 amino acid residues, not greater than 600 amino acid residues, not greater than 500 amino acid residues, not greater than 450 amino acid residues, not greater than 400 amino acid residues, not greater than 350 amino acid residues, not greater than 300 amino acid residues, not greater than 250 amino acid residues, or not greater than 200 amino acid residues.

The term BONT or a particular type or subtype thereof also encompasses their equivalent polynucleotides as well, such as those having certain level (e.g., at least 85%, 90%, 95%, 98%, or 99%) of sequence identity or modified with one or more amino acid residue addition, deletion or substitutions. In some embodiments, the substitutions are conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE 1A
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE 1B
Conservative Amino Acid Substitutions

| For Amino Acid | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, |
| | L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, |
| | Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

In some embodiments, an BoNT peptide includes no more than one, no more than two, or no more than three of the above substitutions from a natural BONT peptide.

Non-limiting examples of BONT light chains include SEQ ID NO: 8 (a BoNT A light chain) and amino sequences having at least 90% (or at least 95%, at least 98% or at least 99%) sequence identity to SEQ ID NO: 8. Non-limiting examples of BoNT heavy chains include SEQ ID NO: 9 (a BoNT A heavy chain) and amino sequences having at least 90% (or at least 95%, at least 98% or at least 99%) sequence identity to SEQ ID NO: 9. The amino acid sequences of SEQ ID NO: 8 and 9 are provided in Table 2 below.

TABLE 2
Representative BoNT sequences

| Name | Sequence | SEQ ID NO: |
|---|---|---|
| BoNTA | MPFVNKQFNYKDPVNGVDIAYIKIPNVGQMQPVKAFKIHNKIWVIPERDT | 8 |
| light | FTNPEEGDLNPPPEAKQVPVSYYDSTYLSTDNEKDNYLKGVTKLFERIYS | |
| chain | TDLGRMLLTSIVRGIPFWGGSTIDTELKVIDTNCINVIQPDGSYRSEELN | |
| | LVIIGPSADIIQFECKSFGHEVLNLTRNGYGSTQYIRFSPDFTFGFEESL | |
| | EVDTNPLLGAGKFATDPAVTLAHELIHAGHRLYGIAINPNRVFKVNTNAY | |
| | YEMSGLEVSFEELRTFGGHDAKFIDSLQENEFRLYYYNKFKDIASTLNKA | |
| | KSIVGTTASLQYMKNVFKEKYLLSEDTSGKFSVDKLKFDKLYKMLTEIYT | |
| | EDNFVKFFKVLNRKTYLNFDKAVFKINIVPKVNYTIYDGENLRNTNLAAN | |
| | FNGQNTEINNMNFTKLKNFTGLFEF | |
| BoNTA | YKLLCVRGIITSKTKSLDKGYNKALNDLCIKVNNWDLFFSPSEDNFTNDL | 9 |
| heavy | NKGEEITSDTNIEAAEENISLDLIQQYYLTFNFDNEPENISIENLSSDII | |
| chain | GQLELMPNIERFPNGKKYELDKYTMFHYLRAQEFEHGKSRIALTNSVNEA | |
| | LLNPSRVYTFFSSDYVKKVNKATEAAMFLGWVEQLVYDFTDETSEVSTTD | |
| | KIADITIIIPYIGPALNIGNMLYKDDFVGALIFSGAVILLEFIPEIAIPV | |
| | LGTFALVSYIANKVLTVQTIDNALSKRNEKWDEVYKYIVTNWLAKVNTQI | |
| | DLIRKKMKEALENQAEATKAIINYQYNQYTEEEKNNINFNIDDLSSKLNE | |
| | SINKAMININKFLNQCSVSYLMNSMIPYGVKRLEDFDASLKDALLKYIYD | |
| | NRGTLIGQVDRLKDKVNNTLSTDIPFQLSKYVDNQRLLSTFTEYIKNIIN | |
| | TSILNLRYESNHLIDLSRYASKINIGSKVNFDPIDKNQIQLENLESSKIE | |
| | VILKNAIVYNSMYENFSTSFWIRIPKYENSISLNNEYTIINCMENNSGWK | |
| | VSLNYGEIIWTLQDTQEIKQRVVFKYSQMINISDYINRWIFVTITNNRLN | |
| | NSKIYINGRLIDQKPISNLGNIHASNNIMFKLDGCRDTHRYIWIKYENLF | |
| | DKELNEKEIKDLYDNQSNSGILKDFWGDYLQYDKPYYMLNLYDPNKYVDV | |
| | NNVGIRGYMYLKGPRGSVMTTNIYLNSSLYRGTKFIIKKYASGNKDNIVR | |
| | NNDRVYINVVVKNKEYRLATNASQAGVEKILSALEIPDVGNLSQVVVMKS | |
| | KNDQGITNKCKMNLQDNNGNDIGFIGFHQFNNIAKLVASNWYNRQIERSS | |
| | RTLGCSWEFIPVDDGWGERPL | |

A “zinc finger motif” is a small protein structural motif that is characterized by the coordination of one or more zinc ions in order to stabilize the fold. In general, zinc fingers coordinate zinc ions with a combination of cysteine and histidine residues. The number and order of these residues can be used to classify different types of zinc fingers (e.g., Cys₂His₂, Cys₄, and Cys₆). Yet another method classifies zinc finger proteins into fold groups based on the overall shape of the protein backbone in the folded domain. The most common fold groups of zinc fingers are the Cys₂His₂ (the classic zinc finger), treble clef, zinc ribbon, gag knuckle, Zn₂/Cys₆, and TAZ2 domain like.

The Cys₂His₂ fold group adopts a simple ββα fold and has the amino acid sequence motif:

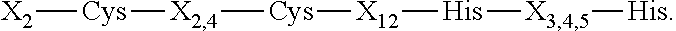

Individual zinc finger domains can occur as tandem repeats with two, three, or more fingers comprising the DNA-binding domain of the protein.

The zinc finger motifs can be modified to remove or reduce their ability to bind to DNA. For instance, a modified Cys₂His₂ contains at least an alanine at residues −1, 2, 3 or 6 of the alpha-helical fragment in the zinc finger motif. Non-limiting examples of zinc finger motifs are shown in Table 3 below. Some of the sequences in Table 3, SEQ ID NO: 1 and 5-7, are individual zinc finger motifs, while a few others (tandem of zinc finger motifs), SEQ ID NO: 2-4, contain multiple concatenated zinc finger motifs. When two or more zinc fingers are used in tandem, they can be located right next to each other or linked via a peptide linker, i.e., a short peptide that is from 1, 2, or 3 amino acid resides to 2, 3, 4, 5, 6, 7, 8, 9 or 10 amino acid residues long). The modified alanine residues of SEQ ID NO: 1 are underlined and bolded.

TABLE 3
Example CPP Sequences

| | | SEQ ID |
|---|---|---|
| CPP | Sequence | NO: |
| C₂H₂ ZFP | EKPYKCPECGKSFSASAALVAHQRTHTG | 1 |
| C₂H₂ ZFP₂ | EKPYKCPECGKSFSASAALVAHQRTHTGEKPYKCPECGKSFSASAALVAHQR | 2 |
| | THTG | |
| C₂H₂ ZFP₃ | EKPYKCPECGKSFSASAALVAHQRTHTGEKPYKCPECGKSFSASAALVAHQR | 3 |
| | THTGEKPYKCPECGKSFSASAALVAHQRTHTG | |
| C₂H₂ ZFP₄ | EKPYKCPECGKSFSASAALVAHQRTHTGEKPYKCPECGKSFSASAALVAHQR | 4 |
| | THTGEKPYKCPECGKSFSASAALVAHQRTHTGEKPYKCPECGKSFSASAALV | |
| | AHQRTHTG | |
| Zn₂/Cys₆ ZFP | RIPLSCTICRKRKVKCDKLRPHCQQCTKTGVAHLCHYM | 5 |
| CCHC ZFP | TCYNCGQTGHLSRECPS | 6 |
| CCHC₅ ZFP | RRCNCMATRHPLFEVAPNCLNCGKIICEKEGLQPCSYCGQELLSPKDKQE | 7 |
| | II | |
| TAT | GRKKRRQRRRPQ | 18 |
| Pep-1 | N-acetyl-KETWWETWWTEWSQPKKKRKV-OH | 19 |
| DPV3 | RKKRRRESRKKRRRES | 20 |
| DPV6 | GRPRESGKKRKRKRLKP | 21 |
| DPV7 | GKRKKKGKLGKKRDP | 22 |
| DPV7b | GKRKKKGKLGKKRPRSR | 23 |
| DPV3/10 | RKKRRRESRRARRSPRHL | 24 |
| DPV10/6 | SRRARRSPRESGKKRKRKR | 25 |
| DPV1047 | VKRGLKLRHVRPRVTRMDV | 26 |
| DPV10 | SRRARRSPRHLGSG | 27 |
| DPV15 | LRRERQSRLRRERQSR | 28 |
| DPV15b | GAYDLRRRERQSRLRRRERQSR | 29 |
| HIV-1 Tat | RKKRRQRRR | 30 |
| FHV coat | RRRRNRTRRNRRRVR | 31 |
| HIV-1 Rev | TRQARRNRRRRWRERQR | 32 |
| HTLV-II Rex | TRRQRTRRARRNR | 33 |
| BMV Gag | KMTRAQRRAAARRNRWTAR | 34 |
| P22 N | NAKTRRHERRRKLAIER | 35 |
| λ(1-22) | MDAQTRRRERRAEKQAQWKAAN | 36 |
| ϕ21N(12-29) | TAKTRYKARRAELIAERR | 37 |
| Yeast PrP6 | TRRNKRNRIQEQLNRK | 38 |
| Protamine 1 | PRRRRSSSRPVRRRRRPRVSRRRRRRGGRRRR | 39 |
| Human cJun | RIKAERKRMRNRIAASKSRKRKLERIAR | 40 |
| Human cFos | KRRIRRERNKMAAAKSRNRRRELTDT | 41 |
| Yeast GCN4 | KRARNTEAARRSRARKLQRMKQ | 42 |
| Penetratin | RQIKIWFQNRRMKWKK | 43 |
| Islet-1 | RVIRVWFQNKRCKDKK | 44 |
| Fushi-tarazu | SKRTRQTYTRYQTLELEKEFHENRYITRRRRIDIANALSLSERQIKIWFQNR | 45 |
| | RMKSKKDR | |
| Engrailed-2 | SQIKIWFQNKRAKIKK | 46 |
| HoxA-13 | RQVTIWFQNRRVKEKK | 47 |
| Knotted-1 | KQINNWFINQRKRHWK | 48 |
| PDX-1 | RHIKIWFQNRRMKWKK | 49 |
| Ig(v) | MGLGLHLLVLAAALQGAKKKRKV | 50 |
| BPrPp(1-30) | MVKSKIGSWILVLFVAMWSDVGLCKKRPKP | 51 |
| MPrPp(1-28) | MANLGYWLLALFVTMWTDVGLCKKRPKP | 52 |
| K-FGF + NLS | AAVLLPVLLAAPVQRKRQKLP | 53 |
| K-FGF | AAVLLPVLLAAP | 54 |
| Bac7 | RRIRPRPPRLPRPRPRPLPFPRPG | 55 |
| Pyrrhocoricin | VDKGSYLPRPTPPRPIYNRN | 56 |
| Human | KCFQWQRNMRKVRGPPVSCIKR | 57 |
| lactoferrin | | |
| (19-40) | | |
| Buforin 2 | TRSSRAGLQWPVGRVHRLLRK | 58 |
| Melittin | GIGAVLKVLTTGLPALISWIKRKRQQ | 59 |
| Magainin 2 | GIGKWLHSAKKFGKAFVGEIMNS | 60 |
| LL-37 | LLGDFFRKSKEKIGKEFKRIVQRIKDELRNLVPRTESC | 61 |
| SynB1 | RGGRLSYSRRRFSTSTGR | 62 |
| Crotamine | YKQCHKKGGKKGSG | 63 |
| S4₁₃-PVᵣₑᵥ | ALWKTLLKKVLKAPKKKRKV | 64 |
| L-2 | HARIKPTFRRLKWKYKGKFW | 65 |
| INV3 | TKRRITPKDVIDVRSVTTEINT | 66 |
| E^{rns} | RQGAARVTSWLGRQLRIAGKRLEGRSK | 67 |
| VP22 | NAATATRGRSAASRPTQRPRAPARSASRPRRPVQ | 68 |
| HIV-1 VPR 77-92 | RHSRIGIIQQRRTRNG | 69 |
| Ribotoxin2 L3 | KLIKGRTPIKFGKADCDRPPKHSQNGMGK | 70 |
| loop | | |
| PreS2-TLM | PLSSIFSRIGDP | 71 |
| VT5 | DPKGDPKGVTVTVTVTVTGKGDPKPD | 72 |
| HRSV | RRIPNRRPRR | 73 |
| AIP6 | RLRWR | 74 |
| ARF(1-22) | MVRRFLVTLRIRRACGPPRVRV | 75 |
| M918 | MVTVLFRRLRIRRACGPPRVRV | 76 |
| pVEC | LLIILRRRIRKQAHAHSK | 77 |
| Azurin p18 | LSTAADMQGVVTDGMASG | 78 |
| Azurin p28 | LSTAADMQGVVTDGMASGLDKDYLKPDD | 79 |
| hCT18-32 | KFHTFPQTAIGVGAP | 80 |
| Bip | VPTLK | 81 |
| Bip | PMLKE | 82 |
| Bip | VPALR | 83 |
| Bip | VSALK | 84 |
| Bip | IPALK | 85 |
| C105Y | PFVYLI | 86 |
| FGF12 | PIEVCMYREP | 87 |
| Polyarginine | RRRRRRRR | 88 |
| Polyarginine | RRRRRRRRR | 89 |
| Polyarginine | RRRRRRRRRR | 90 |
| Polyarginine | RRRRRRRRRRRR | 91 |
| Pep-1 | KETWWETWWTEWSQPKKRKV | 92 |
| MPG | GLAFLGFLGAAGSTMGAWSQPKKKRKV | 93 |
| Transportan | GWTLNSAGYLLGKINLKALAALAKKIL | 94 |
| TH | AGYLLGHINLHHLAHLAibHHIL | 95 |
| MAP | KLALKALKALKAALKLA | 96 |
| W/R | RRWWRRWRR | 97 |
| CADY | GLWRALWRLLRSLWRLLWRA | 98 |
| EB-1 | LIRLWSHLIHIWFQNRRLKWKKK | 99 |
| GALA | WEAALAEALAEALAEHLAEALAEALEALAA | 100 |
| MAP12 | LKTLTETLKELTKTLTEL | 101 |
| MAP17 | QLALQLALQALQAALQLA | 102 |
| (PPR)n | (PPR)₃ | 103 |
| (PPR)n | (PPR)₄ | 104 |
| (PPR)n | (PPR)₅ | 105 |
| (PPR)n | (PPR)₆ | 106 |
| (PRR)n | (PRR)₃ | 107 |
| (PRR)n | (PRR)₄ | 108 |
| (PRR)n | (PRR)₅ | 109 |
| (PRR)n | (PRR)₆ | 110 |
| aPP4R1 | GPSQPTYPGDDAPVRDLIRFYRDLQRYLNVVTRHRY | 111 |
| aPP5R1 | GPSQPTYPGDDAPVRDLIRFYRDLRRYLNVVTRHRY | 112 |
| aPP6R1 | GPSQPTYPGDDAPVRDLRRFYRDLRRYLNVVTRHRY | 113 |
| PoliProline-based | G(P_{L}XX)_{N}P₁ | 114 |
| SAP | VRLPPPVRLPPPVRLPPP | 115 |
| SAP(E) | VELPPPVELPPPVELPPP | 116 |
| SVM1 | FKIYDKKVRTRVVKH | 117 |
| SVM2 | RASKRDGSWVKKLHRILE | 118 |
| SVM3 | KGTYKKKLMRIPLKGT | 119 |
| SVM4 | LYKKGPAKKGRPPLRGWFH | 120 |
| SVM5 | HSPIIPLGTRFVCHGVT | 121 |
| YTA2 | YTAIAWVKAFIRKLRK | 122 |
| YTA4 | IAWVKAFIRKLRKGPLG | 123 |
| SG3 | RLSGMNEVLSFRWL | 124 |
| Pep-7 | SDLWEMMMVSLACQY | 125 |
| | VTWTPQAWFQWV | 126 |
| 439a | GSPWGLQHHPPRT | 127 |
| 435b | GPFHFYQFLFPPV | 128 |
| HN-1 | TSPLNIHNGQKL | 129 |
| BEN_1079 | RCGRASRCRVRWMRRRRI | 130 |
| BEN_0805 | PYSRPHVQLWYPNRESCRSLIRSLGP | 131 |
| Pept1 | PLILLRLLRGQF | 132 |
| Pept2 | PLIYLRLLRGQF | 133 |
| IVV-14 | KLWMRWYSPTTRRYG | 134 |

The distances between the BoNT light chain, heavy and the CPP can be adjusted based on preferences and needs. In some embodiments, a CPP is not longer than 200 amino acid residues away from the N- or C-terminus of an adjacent BONT light or heavy chain. In some embodiments, the distance is from 0 to about 150, from 5 to 100, from 10 to 75, from 10 to 50, from 10 to 40, from 10 to 30, from 10 to 20, from 20 to 150, from 20 to 100, from 20 or 50, or from 50 to 100 amino acid resides. In some embodiments, distance is provided by inserting a spacer sequence (e.g., alanine's, glycine's, or the combinations thereof).

Non-limiting examples of fusion polypeptide sequences are provided in SEQ ID NO: 10 to 17 (Table 4). In some embodiments, the fusion polypeptide is not cleaved (single-chain).

TABLE 4
Representative Chimeric Polypeptide Sequences

| Name | Sequence (with His tag removed) | SEQ ID NO: |
|---|---|---|
| BoNTAFL | MPFVNKQFNYKDPVNGVDIAYIKIPNVGQMQPVK | 10 |
| | AFKIHNKIWVIPERDTFTNPEEGDLNPPPEAKQVPVSYYDSTYLSTDNEK | |
| | DNYLKGVTKLFERIYSTDLGRMLLTSIVRGIPFWGGSTIDTELKVIDTNC | |
| | INVIQPDGSYRSEELNLVIIGPSADIIQFECKSFGHEVLNLTRNGYGSTQ | |
| | YIRFSPDFTFGFEESLEVDTNPLLGAGKFATDPAVTLAHELIHAGHRLYG | |
| | IAINPNRVFKVNTNAYYEMSGLEVSFEELRTFGGHDAKFIDSLQENEFRL | |
| | YYYNKFKDIASTLNKAKSIVGTTASLQYMKNVFKEKYLLSEDTSGKFSVD | |
| | KLKFDKLYKMLTEIYTEDNFVKFFKVLNRKTYLNFDKAVFKINIVPKVNY | |
| | TIYDGFNLRNTNLAANFNGQNTEINNMNFTKLKNFTGLFEFYKLLCVRGI | |
| | ITSKTKSLDKGYNKALNDLCIKVNNWDLFFSPSEDNFTNDLNKGEEITSD | |
| | TNIEAAEENISLDLIQQYYLTFNFDNEPENISIENLSSDIIGQLELMPNI | |
| | ERFPNGKKYELDKYTMFHYLRAQEFEHGKSRIALTNSVNEALLNPSRVYT | |
| | FFSSDYVKKVNKATEAAMFLGWVEQLVYDFTDETSEVSTTDKIADITIII | |
| | PYIGPALNIGNMLYKDDFVGALIFSGAVILLEFIPEIAIPVLGTFALVSY | |
| | IANKVLTVQTIDNALSKRNEKWDEVYKYIVTNWLAKVNTQIDLIRKKMKE | |
| | ALENQAEATKAIINYQYNQYTEEEKNNINFNIDDLSSKLNESINKAMINI | |
| | NKFLNQCSVSYLMNSMIPYGVKRLEDFDASLKDALLKYIYDNRGTLIGQV | |
| | DRLKDKVNNTLSTDIPFQLSKYVDNQRLLSTFTEYIKNIINTSILNLRYE | |
| | SNHLIDLSRYASKINIGSKVNFDPIDKNQIQLENLESSKIEVILKNAIVY | |
| | NSMYENFSTSFWIRIPKYENSISLNNEYTIINCMENNSGWKVSLNYGEII | |
| | WTLQDTQEIKQRVVFKYSQMINISDYINRWIFVTITNNRLNNSKIYINGR | |
| | LIDQKPISNLGNIHASNNIMFKLDGCRDTHRYIWIKYFNLEDKELNEKEI | |
| | KDLYDNQSNSGILKDFWGDYLQYDKPYYMLNLYDPNKYVDVNNVGIRGYM | |
| | YLKGPRGSVMTTNIYLNSSLYRGTKFIIKKYASGNKDNIVRNNDRVYINV | |
| | VVKNKEYRLATNASQAGVEKILSALEIPDVGNLSQVVVMKSKNDQGITNK | |
| | CKMNLQDNNGNDIGFIGFHQFNNIAKLVASNWYNRQIERSSRTLGCSWEF | |
| | IPVDDGWGERPL | |
| His-ZFP3- | EKPYKCPECGKSFSASAALVAHQRTHTGEKPYKC | 11 |
| BoNTALC | PECGKSFSASAALVAHQRTHTGEKPYKCPECGKSFSASAALVAHQRTHTG | |
| | GGSGGSGGSMPFVNKQFNYKDPVNGVDIAYIKIPNVGQMQPVKAFKIHNK | |
| | IWVIPERDTFTNPEEGDLNPPPEAKQVPVSYYDSTYLSTDNEKDNYLKGV | |
| | TKLFERIYSTDLGRMLLTSIVRGIPFWGGSTIDTELKVIDTNCINVIQPD | |
| | GSYRSEELNLVIIGPSADIIQFECKSFGHEVLNLTRNGYGSTQYIRFSPD | |
| | FTFGFEESLEVDTNPLLGAGKFATDPAVTLAHELIHAGHRLYGIAINPNR | |
| | VFKVNTNAYYEMSGLEVSFEELRTFGGHDAKFIDSLQENEFRLYYYNKFK | |
| | DIASTLNKAKSIVGTTASLQYMKNVFKEKYLLSEDTSGKFSVDKLKFDKL | |
| | YKMLTEIYTEDNFVKFFKVLNRKTYLNFDKAVFKINIVPKVNYTIYDGEN | |
| | LRNTNLAANFNGQNTEINNMNFTKLKNFTGLFEF | |
| His-ZFP3- | EKPYKCPECGKSFSASAALVAHQRTHTGEKPYKC | 12 |
| BoNTAFL | PECGKSFSASAALVAHQRTHTGEKPYKCPECGKSFSASAALVAHQRTHTG | |
| | GGSGGSGGSMPFVNKQFNYKDPVNGVDIAYIKIPNVGQMQPVKAFKIHNK | |
| | IWVIPERDTFTNPEEGDLNPPPEAKQVPVSYYDSTYLSTDNEKDNYLKGV | |
| | TKLFERIYSTDLGRMLLTSIVRGIPFWGGSTIDTELKVIDTNCINVIQPD | |
| | GSYRSEELNLVIIGPSADIIQFECKSFGHEVLNLTRNGYGSTQYIRFSPD | |
| | FTFGFEESLEVDTNPLLGAGKFATDPAVTLAHELIHAGHRLYGIAINPNR | |
| | VFKVNTNAYYEMSGLEVSFEELRTFGGHDAKFIDSLQENEFRLYYYNKFK | |
| | DIASTLNKAKSIVGTTASLQYMKNVFKEKYLLSEDTSGKFSVDKLKEDKL | |
| | YKMLTEIYTEDNFVKFFKVLNRKTYLNFDKAVFKINIVPKVNYTIYDGEN | |
| | LRNTNLAANFNGQNTEINNMNFTKLKNFTGLFEFYKLLCVRGIITSKTKS | |
| | LDKGYNKALNDLCIKVNNWDLFFSPSEDNFTNDLNKGEEITSDTNIEAAE | |
| | ENISLDLIQQYYLTFNFDNEPENISIENLSSDIIGQLELMPNIERFPNGK | |
| | KYELDKYTMFHYLRAQEFEHGKSRIALTNSVNEALLNPSRVYTFFSSDYV | |
| | KKVNKATEAAMFLGWVEQLVYDFTDETSEVSTTDKIADITIIIPYIGPAL | |
| | NIGNMLYKDDFVGALIFSGAVILLEFIPEIAIPVLGTFALVSYIANKVLT | |
| | VQTIDNALSKRNEKWDEVYKYIVTNWLAKVNTQIDLIRKKMKEALENQAE | |
| | ATKAIINYQYNQYTEEEKNNINFNIDDLSSKLNESINKAMININKELNQC | |
| | SVSYLMNSMIPYGVKRLEDFDASLKDALLKYIYDNRGTLIGQVDRLKDKV | |
| | NNTLSTDIPFQLSKYVDNQRLLSTFTEYIKNIINTSILNLRYESNHLIDL | |
| | SRYASKINIGSKVNFDPIDKNQIQLENLESSKIEVILKNAIVYNSMYENF | |
| | STSFWIRIPKYENSISLNNEYTIINCMENNSGWKVSLNYGEIIWTLQDTQ | |
| | EIKQRVVFKYSQMINISDYINRWIFVTITNNRLNNSKIYINGRLIDQKPI | |
| | SNLGNIHASNNIMFKLDGCRDTHRYIWIKYENLFDKELNEKEIKDLYDNQ | |
| | SNSGILKDFWGDYLQYDKPYYMLNLYDPNKYVDVNNVGIRGYMYLKGPRG | |
| | SVMTTNIYLNSSLYRGTKFIIKKYASGNKDNIVRNNDRVYINVVVKNKEY | |
| | RLATNASQAGVEKILSALEIPDVGNLSQVVVMKSKNDQGITNKCKMNLQD | |
| | NNGNDIGFIGFHQFNNIAKLVASNWYNRQIERSSRTLGCSWEFIPVDDGW | |
| | GERPL | |
| BoNTALC- | MPFVNKQFNYKDPVNGVDIAYIKIPNVGQMQPVKAFKIHNKIWVIPERDT | 13 |
| ZFP3-His | FTNPEEGDLNPPPEAKQVPVSYYDSTYLSTDNEKDNYLKGVTKLFERIYS | |
| | TDLGRMLLTSIVRGIPFWGGSTIDTELKVIDTNCINVIQPDGSYRSEELN | |
| | LVIIGPSADIIQFECKSFGHEVLNLTRNGYGSTQYIRFSPDFTFGFEESL | |
| | EVDTNPLLGAGKFATDPAVTLAHELIHAGHRLYGIAINPNRVFKVNTNAY | |
| | YEMSGLEVSFEELRTFGGHDAKFIDSLQENEFRLYYYNKFKDIASTLNKA | |
| | KSIVGTTASLQYMKNVFKEKYLLSEDTSGKFSVDKLKFDKLYKMLTEIYT | |
| | EDNFVKFFKVLNRKTYLNFDKAVFKINIVPKVNYTIYDGENLRNTNLAAN | |
| | FNGQNTEINNMNFTKLKNFTGLFEFGGSGGSGGSEKPYKCPECGKSFSAS | |
| | AALVAHQRTHTGEKPYKCPECGKSFSASAALVAHQRTHTGEKPYKCPECG | |
| | KSFSASAALVAHQRTHT | |
| BoNTAFL- | MPFVNKQFNYKDPVNGVDIAYIKIPNVGQMQPVKAFKIHNKIWVIPERDT | 14 |
| ZFP3-His | FTNPEEGDLNPPPEAKQVPVSYYDSTYLSTDNEKDNYLKGVTKLFERIYS | |
| | TDLGRMLLTSIVRGIPFWGGSTIDTELKVIDTNCINVIQPDGSYRSEELN | |
| | LVIIGPSADIIQFECKSFGHEVLNLTRNGYGSTQYIRFSPDFTFGFEESL | |
| | EVDTNPLLGAGKFATDPAVTLAHELIHAGHRLYGIAINPNRVFKVNTNAY | |
| | YEMSGLEVSFEELRTFGGHDAKFIDSLQENEFRLYYYNKFKDIASTINKA | |
| | KSIVGTTASLQYMKNVFKEKYLLSEDTSGKFSVDKLKFDKLYKMLTEIYT | |
| | EDNFVKFFKVLNRKTYLNFDKAVFKINIVPKVNYTIYDGFNLRNTNLAAN | |
| | FNGQNTEINNMNFTKLKNFTGLFEFYKLLCVRGIITSKTKSLDKGYNKAL | |
| | NDLCIKVNNWDLFFSPSEDNFTNDLNKGEEITSDTNIEAAEENISLDLIQ | |
| | QYYLTFNFDNEPENISIENLSSDIIGQLELMPNIERFPNGKKYELDKYTM | |
| | FHYLRAQEFEHGKSRIALTNSVNEALLNPSRVYTFFSSDYVKKVNKATEA | |
| | AMFLGWVEQLVYDFTDETSEVSTTDKIADITIIIPYIGPALNIGNMLYKD | |
| | DFVGALIFSGAVILLEFIPEIAIPVLGTFALVSYIANKVLTVQTIDNALS | |
| | KRNEKWDEVYKYIVTNWLAKVNTQIDLIRKKMKEALENQAEATKAIINYQ | |
| | YNQYTEEEKNNINFNIDDLSSKLNESINKAMININKFLNQCSVSYLMNSM | |
| | IPYGVKRLEDFDASLKDALLKYIYDNRGTLIGQVDRLKDKVNNTLSTDIP | |
| | FQLSKYVDNQRLLSTFTEYIKNIINTSILNLRYESNHLIDLSRYASKINI | |
| | GSKVNFDPIDKNQIQLENLESSKIEVILKNAIVYNSMYENFSTSFWIRIP | |
| | KYFNSISLNNEYTIINCMENNSGWKVSLNYGEIIWTLQDTQEIKQRVVFK | |
| | YSQMINISDYINRWIFVTITNNRLNNSKIYINGRLIDQKPISNLGNIHAS | |
| | NNIMFKLDGCRDTHRYIWIKYFNLEDKELNEKEIKDLYDNQSNSGILKDF | |
| | WGDYLQYDKPYYMLNLYDPNKYVDVNNVGIRGYMYLKGPRGSVMTTNIYL | |
| | NSSLYRGTKFIIKKYASGNKDNIVRNNDRVYINVVVKNKEYRLATNASQA | |
| | GVEKILSALEIPDVGNLSQVVVMKSKNDQGITNKCKMNLQDNNGNDIGFI | |
| | GFHQFNNIAKLVASNWYNRQIERSSRTLGCSWEFIPVDDGWGERPLGGSG | |
| | GSGGSEKPYKCPECGKSFSASAALVAHQRTHTGEKPYKCPECGKSESASA | |
| | ALVAHQRTHTGEKPYKCPECGKSFSASAALVAHQRTHT | |
| His-ZFP3- | EKPYKCPECGKSFSASAALVAHQRTHTGEKPYKC | 15 |
| BoNTAFL- | PECGKSFSASAALVAHQRTHTGEKPYKCPECGKSFSASAALVAHQRTHTG | |
| ZFP3 | GGSGGSGGSMPFVNKQFNYKDPVNGVDIAYIKIPNVGQMQPVKAFKIHNK | |
| | IWVIPERDTFTNPEEGDLNPPPEAKQVPVSYYDSTYLSTDNEKDNYLKGV | |
| | TKLFERIYSTDLGRMLLTSIVRGIPFWGGSTIDTELKVIDTNCINVIQPD | |
| | GSYRSEELNLVIIGPSADIIQFECKSFGHEVLNLTRNGYGSTQYIRFSPD | |
| | FTFGFEESLEVDTNPLLGAGKFATDPAVTLAHELIHAGHRLYGIAINPNR | |
| | VFKVNTNAYYEMSGLEVSFEELRTFGGHDAKFIDSLQENEFRLYYYNKFK | |
| | DIASTLNKAKSIVGTTASLQYMKNVFKEKYLLSEDTSGKFSVDKLKFDKL | |
| | YKMLTEIYTEDNFVKFFKVLNRKTYLNFDKAVFKINIVPKVNYTIYDGEN | |
| | LRNTNLAANFNGQNTEINNMNFTKLKNFTGLFEFYKLLCVRGIITSKTKS | |
| | LDKGYNKALNDLCIKVNNWDLFFSPSEDNFTNDLNKGEEITSDTNIEAAE | |
| | ENISLDLIQQYYLTFNFDNEPENISIENLSSDIIGQLELMPNIERFPNGK | |
| | KYELDKYTMFHYLRAQEFEHGKSRIALTNSVNEALLNPSRVYTFFSSDYV | |
| | KKVNKATEAAMFLGWVEQLVYDFTDETSEVSTTDKIADITIIIPYIGPAL | |
| | NIGNMLYKDDFVGALIFSGAVILLEFIPEIAIPVLGTFALVSYIANKVLT | |
| | VQTIDNALSKRNEKWDEVYKYIVTNWLAKVNTQIDLIRKKMKEALENQAE | |
| | ATKAIINYQYNQYTEEEKNNINFNIDDLSSKLNESINKAMININKELNQC | |
| | SVSYLMNSMIPYGVKRLEDFDASLKDALLKYIYDNRGTLIGQVDRLKDKV | |
| | NNTLSTDIPFQLSKYVDNQRLLSTFTEYIKNIINTSILNLRYESNHLIDL | |
| | SRYASKINIGSKVNFDPIDKNQIQLENLESSKIEVILKNAIVYNSMYENF | |
| | STSFWIRIPKYENSISLNNEYTIINCMENNSGWKVSLNYGEIIWTLQDTQ | |
| | EIKQRVVFKYSQMINISDYINRWIFVTITNNRLNNSKIYINGRLIDQKPI | |
| | SNLGNIHASNNIMFKLDGCRDTHRYIWIKYENLFDKELNEKEIKDLYDNQ | |
| | SNSGILKDFWGDYLQYDKPYYMLNLYDPNKYVDVNNVGIRGYMYLKGPRG | |
| | SVMTTNIYLNSSLYRGTKFIIKKYASGNKDNIVRNNDRVYINVVVKNKEY | |
| | RLATNASQAGVEKILSALEIPDVGNLSQVVVMKSKNDQGITNKCKMNLQD | |
| | NNGNDIGFIGFHQFNNIAKLVASNWYNRQIERSSRTLGCSWEFIPVDDGW | |
| | GERPLGGSGGSGGSEKPYKCPECGKSFSASAALVAHQRTHTGEKPYKCPE | |
| | CGKSFSASAALVAHQRTHTGEKPYKCPECGKSFSASAALVAHQRTHTG | |
| His-TAT- | GRKKRRQRRRPQGGSGGSGGSMPFVNKQFNYKDP | 16 |
| BoNTAFL | VNGVDIAYIKIPNVGQMQPVKAFKIHNKIWVIPERDTFTNPEEGDLNPPP | |
| | EAKQVPVSYYDSTYLSTDNEKDNYLKGVTKLFERIYSTDLGRMLLTSIVR | |
| | GIPFWGGSTIDTELKVIDTNCINVIQPDGSYRSEELNLVIIGPSADIIQF | |
| | ECKSFGHEVLNLTRNGYGSTQYIRFSPDFTFGFEESLEVDTNPLLGAGKF | |
| | ATDPAVTLAHELIHAGHRLYGIAINPNRVFKVNTNAYYEMSGLEVSFEEL | |
| | RTFGGHDAKFIDSLQENEFRLYYYNKFKDIASTLNKAKSIVGTTASLQYM | |
| | KNVFKEKYLLSEDTSGKFSVDKLKFDKLYKMLTEIYTEDNFVKFFKVLNR | |
| | KTYLNFDKAVFKINIVPKVNYTIYDGENLRNTNLAANENGQNTEINNMNF | |
| | TKLKNFTGLFEFYKLLCVRGIITSKTKSLDKGYNKALNDLCIKVNNWDLF | |
| | FSPSEDNFTNDLNKGEEITSDTNIEAAEENISLDLIQQYYLTFNFDNEPE | |
| | NISIENLSSDIIGQLELMPNIERFPNGKKYELDKYTMFHYLRAQEFEHGK | |
| | SRIALTNSVNEALLNPSRVYTFFSSDYVKKVNKATEAAMFLGWVEQLVYD | |
| | FTDETSEVSTTDKIADITIIIPYIGPALNIGNMLYKDDFVGALIFSGAVI | |
| | LLEFIPEIAIPVLGTFALVSYIANKVLTVQTIDNALSKRNEKWDEVYKYI | |
| | VTNWLAKVNTQIDLIRKKMKEALENQAEATKAIINYQYNQYTEEEKNNIN | |
| | FNIDDLSSKLNESINKAMININKFLNQCSVSYLMNSMIPYGVKRLEDFDA | |
| | SLKDALLKYIYDNRGTLIGQVDRLKDKVNNTLSTDIPFQLSKYVDNQRLL | |
| | STFTEYIKNIINTSILNLRYESNHLIDLSRYASKINIGSKVNFDPIDKNQ | |
| | IQLFNLESSKIEVILKNAIVYNSMYENFSTSFWIRIPKYENSISLNNEYT | |
| | IINCMENNSGWKVSLNYGEIIWTLQDTQEIKQRVVFKYSQMINISDYINR | |
| | WIFVTITNNRLNNSKIYINGRLIDQKPISNLGNIHASNNIMFKLDGCRDT | |
| | HRYIWIKYFNLEDKELNEKEIKDLYDNQSNSGILKDFWGDYLQYDKPYYM | |
| | LNLYDPNKYVDVNNVGIRGYMYLKGPRGSVMTTNIYLNSSLYRGTKFIIK | |
| | KYASGNKDNIVRNNDRVYINVVVKNKEYRLATNASQAGVEKILSALEIPD | |
| | VGNLSQVVVMKSKNDQGITNKCKMNLQDNNGNDIGFIGFHQFNNIAKLVA | |
| | SNWYNRQIERSSRTLGCSWEFIPVDDGWGERPL | |
| His-Pep1- | KETWWETWWTEWSQPKKKRKVGGSGGSGGSMPFV | 17 |
| BoNTAFL | NKQFNYKDPVNGVDIAYIKIPNVGQMQPVKAFKIHNKIWVIPERDTFTNP | |
| | EEGDLNPPPEAKQVPVSYYDSTYLSTDNEKDNYLKGVTKLFERIYSTDLG | |
| | RMLLTSIVRGIPFWGGSTIDTELKVIDTNCINVIQPDGSYRSEELNLVII | |
| | GPSADIIQFECKSFGHEVLNLTRNGYGSTQYIRFSPDFTFGFEESLEVDT | |
| | NPLLGAGKFATDPAVTLAHELIHAGHRLYGIAINPNRVFKVNTNAYYEMS | |
| | GLEVSFEELRTFGGHDAKFIDSLQENEFRLYYYNKFKDIASTLNKAKSIV | |
| | GTTASLQYMKNVFKEKYLLSEDTSGKFSVDKLKFDKLYKMLTEIYTEDNF | |
| | VKFFKVLNRKTYLNFDKAVFKINIVPKVNYTIYDGENLRNTNLAANENGQ | |
| | NTEINNMNFTKLKNFTGLFEFYKLLCVRGIITSKTKSLDKGYNKALNDLC | |
| | IKVNNWDLFFSPSEDNFTNDLNKGEEITSDTNIEAAEENISLDLIQQYYL | |
| | TFNFDNEPENISIENLSSDIIGQLELMPNIERFPNGKKYELDKYTMFHYL | |
| | RAQEFEHGKSRIALTNSVNEALLNPSRVYTFFSSDYVKKVNKATEAAMEL | |
| | GWVEQLVYDFTDETSEVSTTDKIADITIIIPYIGPALNIGNMLYKDDFVG | |
| | ALIFSGAVILLEFIPEIAIPVLGTFALVSYIANKVLTVQTIDNALSKRNE | |
| | KWDEVYKYIVTNWLAKVNTQIDLIRKKMKEALENQAEATKAIINYQYNQY | |
| | TEEEKNNINFNIDDLSSKLNESINKAMININKFLNQCSVSYLMNSMIPYG | |
| | VKRLEDFDASLKDALLKYIYDNRGTLIGQVDRLKDKVNNTLSTDIPFQLS | |
| | KYVDNQRLLSTFTEYIKNIINTSILNLRYESNHLIDLSRYASKINIGSKV | |
| | NFDPIDKNQIQLFNLESSKIEVILKNAIVYNSMYENFSTSFWIRIPKYEN | |
| | SISLNNEYTIINCMENNSGWKVSLNYGEIIWTLQDTQEIKQRVVFKYSQM | |
| | INISDYINRWIFVTITNNRLNNSKIYINGRLIDQKPISNLGNIHASNNIM | |
| | FKLDGCRDTHRYIWIKYFNLFDKELNEKEIKDLYDNQSNSGILKDFWGDY | |
| | LQYDKPYYMLNLYDPNKYVDVNNVGIRGYMYLKGPRGSVMTTNIYLNSSL | |
| | YRGTKFIIKKYASGNKDNIVRNNDRVYINVVVKNKEYRLATNASQAGVEK | |
| | ILSALEIPDVGNLSQVVVMKSKNDQGITNKCKMNLQDNNGNDIGFIGFHQ | |
| | FNNIAKLVASNWYNRQIERSSRTLGCSWEFIPVDDGWGERPL | |

In some embodiments, the polypeptides may be conjugated to therapeutic agents, prodrugs, peptides, proteins, enzymes, viruses, lipids, biological response modifiers, pharmaceutical agents, or PEG. The polypeptides may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The polypeptides can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The polypeptides can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The present disclosure also provides isolated polynucleotides or nucleic acid molecules encoding the polypeptides, variants or derivatives thereof of the disclosure. The polynucleotides of the present disclosure may encode the entire heavy and light chain of the polypeptides, variants or derivatives thereof on the same polynucleotide molecule or on separate polynucleotide molecules. Additionally, the polynucleotides of the present disclosure may encode portions of the heavy and light chain of the polypeptides, variants or derivatives thereof on the same polynucleotide molecule or on separate polynucleotide molecules.

Polynucleotides encoding a fusion polypeptide or domains thereof can be inserted into an “expression vector”. The term “expression vector” refers to a genetic construct such as a plasmid, virus or other vehicle known in the art that can be engineered to contain a polynucleotide encoding a polypeptide of the disclosure. Such expression vectors are typically plasmids that contain a promoter sequence that facilitates transcription of the inserted genetic sequence in a host cell. The expression vector typically contains an origin of replication, and a promoter, as well as genes that allow phenotypic selection of the transformed cells (e.g., an antibiotic resistance gene). Various promoters, including inducible and constitutive promoters, can be utilized in the disclosure. Typically, the expression vector contains a replicon site and control sequences that are derived from a species compatible with the host cell.

Transformation or transfection of a host cell with a polynucleotide can be carried out using conventional techniques well known to those skilled in the art. For example, where the host cell is E. coli, competent cells that are capable of DNA uptake can be prepared using the CaCl₂, MgCl₂ or RbCl methods known in the art. Alternatively, physical means, such as electroporation or microinjection can be used. Electroporation allows transfer of a polynucleotide into a cell by high voltage electric impulse. Additionally, polynucleotides can be introduced into host cells by protoplast fusion, using methods well known in the art. Suitable methods for transforming eukaryotic cells, such as electroporation and lipofection, also are known.

“Host cells” encompassed by of the disclosure are any cells in which the polynucleotides of the disclosure can be used to express the fusion polypeptide or functional domains thereof. The term also includes any progeny of a host cell. Host cells, which are useful, include bacterial cells (e.g., Clostridium botulinum), fungal cells (e.g., yeast cells), insect cells (e.g., Spodoptera), plant cells and animal cells. A fusion polypeptide of the disclosure can be produced by expression of polynucleotide encoding a fusion polypeptide in prokaryotes. These include, but are not limited to, microorganisms, such as bacteria transformed with recombinant bacteriophage DNA, plasmid DNA, or cosmid DNA expression vectors encoding a fusion polypeptide of the disclosure. Introduction of the construct into the host cell can be effected by calcium phosphate transfection, DEAE-Dextran mediated transfection, or electroporation.

The constructs can be expressed in Clostridium botulinum, which is where BoNT proteins are naturally produced. It is a surprising discovery of the present disclosure that the chimeric proteins containing a BoNT light chain and/or heavy chain can be efficiently produced in insect cells (e.g., Spodoptera frugiperda Sf9). Accordingly, in one embodiment, the host cell can be an insect cell, such as a Lepidoptera cell, a Noctuidae cell, a Spodoptera cell, and a Spodoptera frugiperda cell.

For long-term, high-yield production of recombinant proteins, stable expression is typically used. Rather than using expression vectors that contain viral origins of replication, host cells can be transformed with the cDNA encoding a fusion polypeptide of the disclosure controlled by appropriate expression control elements (e.g., promoter, enhancer, sequences, transcription terminators, polyadenylation sites, and the like), and a selectable marker. The selectable marker confers resistance to a selective killing agent and upon stable integration of the heterologous polynucleotide, allows growth of resistant cells. Such resistant cells grow to form foci that, in turn, can be cloned and expanded into cell lines.

As described herein, the fusion polypeptides of the present disclosure can be effectively delivered via intramuscular injection, which provide excellent efficacy and greatly improved therapeutic index.

In addition, as demonstrated in the examples, the fusion polypeptides have increased duration than the wild-type after repeated dosing. In some embodiments, therefore, the fusion polypeptides are intramuscularly administered no more than once every 4, 8, 12, 16, or 24 weeks, or every 1, 2, 3, 4, 5, 6, 7, 8, 9, 10, 11 or 12 months.

The methods here have broad cosmetic and therapeutic applications. For cosmetic applications, they can be useful for treating wrinkles, adjusting the corners of the mouth or lines from the upper lips. In therapeutics, they can be useful for treating neurological disorders such as dystonias, sparsticity, hemifacial spasm, hyperhidrosis (excessive sweating), hypersalivation (excessive saliva). The methods may also be used for urological disorders such as detrusor sphincter dyssynergia, idiopathic detrusor overactivity, neurogenic detrusor overactivity, urinary retention, anal fissures, benign prostate hyperplasia. Still further indications gastroenterological, otolaryngological disorders or other medical conditions. In some embodiments, the methods are used for treating facial wrinkle, dystonias, sparsticity, hemifacial spasm, hyperhidrosis, or hypersalivation.

A specific dosage and treatment regimen for any particular patient will depend upon a variety of factors, including the particular polypeptides, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

### Compositions

The present disclosure also provides pharmaceutical compositions and formulation suitable for intramuscular administration. Such compositions/formulations comprise an effective amount of a CPP-BONT fusion polypeptide, and an acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of gels, creams, sprays, solutions, suspensions, emulsion, tablets, pills, capsules, powders, sustained-release formulations and the like. The composition can be formulated as a suppository, with traditional binders and carriers such as triglycerides.

In accordance with one embodiment of the present disclosure, therefore, provided is a pharmaceutical composition or formulation comprising a polypeptide comprising a BoNT fused to a cell penetrating peptide (CPP), wherein the BoNT comprises a light chain and a heavy chain.

Non-limiting examples of CPPs are provided in Table 3. One example type of CPP is zinc finger proteins which are naturally occurring transcription factors and can be reprogrammed to recognize targeted genomic loci. Another example is TAT (GRKKRRQRRRPQ, SEQ ID NO: 18, or simply RKKRRQRRR, SEQ ID NO: 30) is derived from the transactivator of transcription (TAT) of human immunodeficiency virus. Yet another example is Pep-1 (N-acetyl-KETWWETWWTEWSQPKKKRKV-OH, SEQ ID NO: 19) which is an amphipathic CPP—a first part is hydrophobic and contains several tryptophan residues (W) that can be involved in membrane destabilization processes, and a second part is cationic with lysine and arginine residues.

In some embodiments, the BoNT protein being administered or formulated is a single-chain protein, or at least a substantial portion of the composition being administered or formulated is single-chain, which is unexpected found to be active in the form of the CPP-BONT fusion protein. In some embodiments, at least 50%, 55%, 60%, 65%, 70%, 75%, 80%, 85%, 90%, 95%, 96%, 97%, 98%, 99%, 99.5%, 99.8%, 99.9% or all of the BoNT proteins are single-chain. In some embodiments, at least 50%, 55%, 60%, 65%, 70%, 75%, 80%, 85%, 90%, 95%, 96%, 97%, 98%, 99%, 99.5%, 99.8%, 99.9% or all of the BoNT proteins are single-chain and are not capable of cleaving itself into two chains.

In some embodiments, the single-chain BoNT proteins are produced from insect cells. Example insect cells include Lepidoptera cells, Noctuidae cells, Spodoptera cells, and Spodoptera frugiperda cells. It is contemplated that the insect production system generates a BONT protein that is different from those produced from prokaryotic cells in terms of protein folding or post-translational modification. These proteins, therefore, are unable of self-cleavage.

In some embodiments, the BoNT included in the CPP-BONT fusion is a mutant BONT with one or more mutations that disable the cleavage. For instance, for BONTA, amino acid residues Lys438-Ala449 (residue position according to Protein ID 1) may be mutated to a different amino acid. In some embodiments, the mutation is a non-conservative mutation. Example mutations include, without limitation, HTQSLDQGYNDDDDKA (SEQ ID NO: 136) and HTQSLDQGGENLYFQGA (SEQ ID NO: 137).

In some embodiments, the CPP is located at the N-terminal side of the BoNT protein. In some embodiments, the CPP is located at the C-terminal side of the BoNT protein. In some embodiments, at least one CPP is located at the N-terminal side of the BoNT protein, and at least one CPP is located at the C-terminal side of the BoNT protein. In some embodiments, at either or both of the N-terminal and C-terminal sides, there are more than one CPP molecule.

In some embodiments, the formulation is lyophilized. In some embodiments, the formulation is an injectable aqueous solution. In some embodiments, the formulation is packaged in a cartridge or vial.

Kits and packages are also provided in certain embodiments that includes a composition or formulation thereof, and instructions for using the composition or formulation. In some embodiments, the kit or package further includes a needle for delivering the composition or formulation.

### EXAMPLES

### Example 1. Expression and Activity of BoNTA-CPP Fusion Proteins from Escherichia coli

This experiment demonstrates that BONTA-CPP (cell penetration peptide) fusion proteins can be expressed and purified from E. coli cells, which are capable of cleaving botulinum substrate SNAP-25, and uptake by cells.

### Methods

### Protein Expression

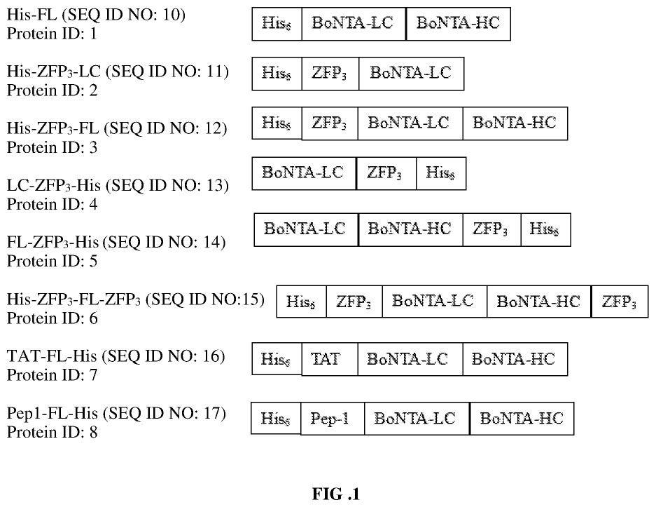

pET28b vectors containing different BoNTA constructs (Protein ID: 1˜8; FIG. 1) were transformed into BL21(DE3) E. coli cells. The sequences of these constructs are provided in SEQ ID NO: 10-17. His₆: histidine tag; BONTA-LC: BONT A light chain; BONTA-HC: BONT A heavy chain; ZFP₂: tandem zinc finger peptides (SEQ ID NO: 1); TAT: transactivator of transcription peptide; Pep-1: pep-1 peptide. TAT and Pep-1 are known cell penetration peptides (CPP).

A single colony was picked from the agar plate and grown in 10 mL lysogeny broth (LB) medium supplemented with 50 μg/mL kanamycin and 90 μM ZnCl₂ at 37° C. overnight. The next day, 10 mL of the starter culture was inoculated into 1 liter LB medium supplemented with 50 μg/mL kanamycin and 90 μM ZnCl₂ and grown to an OD₆₀₀ of 0.8. Protein expression was induced with 0.1 mM isopropyl-ß-D-thiogalactopyranoside (IPTG) at 25° C. for 4 h. Cell pellet was harvested by centrifugation at 5,000 rpm for 10 min.

### Protein Purification

Cell pellet from 2 liter culture (approximately 30 gram) was resuspended in 200 mL BONTA lysis buffer (20 mM HEPES, pH 7.0, 500 mM NaCl, 0.01% Triton X-100, 1× protease cocktail (Roche) and 10% glycerol) and sonicated on ice for three times. Lysed cells were centrifuged at 25,000 g for 1 h at 4° C. and the supernatant was transferred to a new tube. To this supernatant was added 1 mL (settled volume) equilibrated Ni-NTA resins (Qiagen). BONTA proteins containing His₆ tag was allowed to bind with the resins for 30 min with rotation. The resins were transferred to a column and the flow-through was discarded. The resins were washed with 50 mL BoNTA wash buffer (20 mM HEPES, pH 7.0, 500 mM NaCl and 10% glycerol, 20 mM imidazole) and then eluted with 50 mL BoNTA elution buffer (20 mM HEPES, pH 7.0, 500 mM NaCl and 10% glycerol, 300 mM imidazole). The elution fractions were then concentrated and then further purified by ion exchange using start buffer (20 mM TrisHCl, pH 8.5) and end buffer (20 mM TrisHCl, pH 8.5, 1 M NaCl). The fractions with optimum purity were recombined, concentrated by spin concentrated, supplemented with 10% glycerol and then stored at −80° C. for further application.

### Botulinum Activity Test Using SNAPtide™ Assay

SNAPtide (Millipore, Cat. No. 567333-200NMOL) was diluted into 5 μM with reaction buffer (20 mM, pH 7.4, 0.25 mM ZnCl₂, 5 mM DTT, 0.05% Tween-20). Each of the recombinant BoNTA-ZFP fusion proteins (200 nM; Protein ID: 1˜8) was added into reaction buffer containing SNAPtide. The reaction was incubated at 37° C. for 40 min. The fluorescence was recorded using a plate reader with an excitation wavelength of 320 nm and an emission wavelength of 420 nm. The SNAPtide was a short peptide derived from SNAP-25, the intracellular substrate of BONTA. SNAPtide contained the cleavage site of BONTA and both a fluorophore and a quencher groups. Cleavage of the peptide frees the fluorophore and activates fluorescence. Reaction positive control was a commercially available recombinant BoNTA light chain (BoNTA-LC) protein purchased from R&D Systems (Cat. No. 4489-ZN-010). All data were performed in three replicates.

Transduction of hDF Cells with Recombinant BONTA-ZFP Fusion Proteins

hDF cells were seeded on to 6-well plates pre-coated with poly-lysine. At 24 h after seeding, cells were washed with phosphate buffered saline (PBS) for three times. BONTA-ZFP proteins (Protein ID 5 and 6) and control protein (R&D BONTA-LC, a commercially available light chain of BoNTA from R&D Systems) were diluted with DMEM serum-free medium. Cells were treated with BoNTA-ZFP proteins (0.15 μM) and BONTA-LC (0.5 μM) at 37° C. for 2 h. The cells were then washed three times with PBS supplemented with 0.5 mg/mL heparin to remove surface-bound proteins and then harvested by trypsin treatment. The collected cells were lysed by sonication and the BoNTA activity was assayed as described above.

### Results and Data Analysis

One-step affinity purification yielded proteins with only modest purity. After the second-step ion exchange, the purity was largely enhanced. The overall yield was estimated to be 0.1 mg per liter culture and the purity of the final products was 90%.

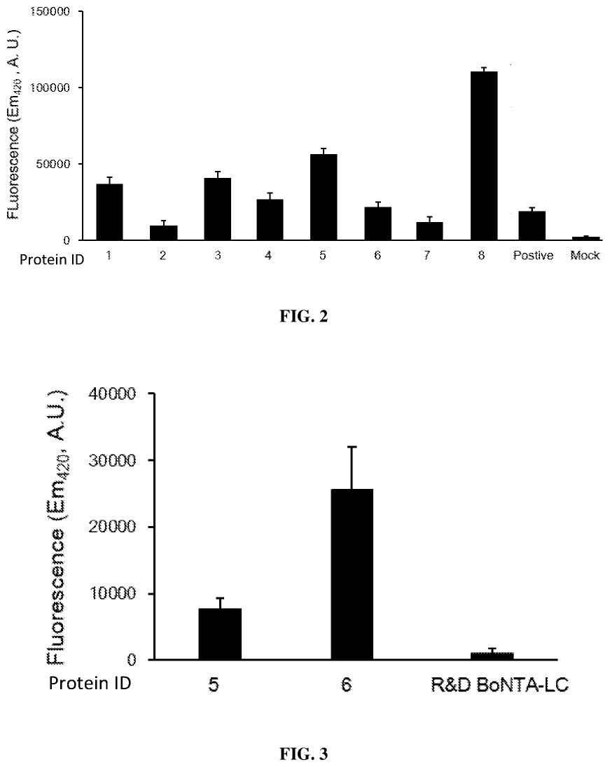

The activity data in FIG. 2 are shown as mean±standard deviation and analyzed by one-tailed Student's t test. All tested samples, including the commercial BONTA-LC, have significantly (p<0.05) higher signals than SNAPtide control group. As shown in FIG. 3, both single-end and bipartite fusion of ZFP retained the activity of BoNTA on the peptide substrate derived from SNAP-25.

hDF cells treated with BoNTA-ZFP proteins, in particular Protein ID 6, exhibited evident BONTA activity, with significantly higher signals than the control group (p<0.05). This demonstrates that BoNTA-ZFP fusions could penetrate cells effectively.

### Example 2. In Vivo Activity of BoNTA-ZFP Proteins in Mice

This experiment demonstrates that, when applied to intact or microneedle-treated mouse skin, BoNTA-ZFP fusion proteins can cause muscle paralysis, characterized by abduction of digits.

### Digit Abduction Experiments

Fifteen C57 female mice with a weight of approximately 36 g were randomly divided into 5 groups (n=3). In all mice, the left legs were left untreated as a control and the right legs were treated with different drugs. Mice were anaesthetized before treatment. In mock group (A), mice were administrated with storage buffer (20 mM HEPES, pH 7.0, 300 mM NaCl and 10% glycerol). In BOTOX (Allergan) injection group (B), BOTOX was reconstituted with 0.9% NaCl saline as instructed and 5 μL of 45 U/mL solution was injected into the right legs. In group C, mice legs and feet were pre-treated with microneedle roller (RoHS MR20, 0.2 mm, house use) and then 60 μL of 45 U/mL BOTOX was topically applied. In group D, mice legs and feet were pre-treated with microneedle roller (RoHS MR20, 0.2 mm, house use) and then 60 μL of 0.05 mg/mL ZFP₃-BONTA-ZFP₃ protein (Protein ID 6) in storage buffer (20 mM HEPES, pH 7.0, 300 mM NaCl and 10% glycerol) was topically applied. In group E, 60 μL of 0.05 mg/mL BONTA-ZFP protein in storage buffer (20 mM HEPES, pH 7.0, 300 mM NaCl and 10% glycerol) was topically applied. Microneedle roller treatment was applied by rolling three times on legs and feet. When topically applied, legs and feet were uniformly spread with substances, massaged and then air dried, which was repeated for several times until all solution was administrated. Digit abduction was recorded after mice were awake at Day 0 and then recorded each day for the following four days.

### Results and Data Analysis

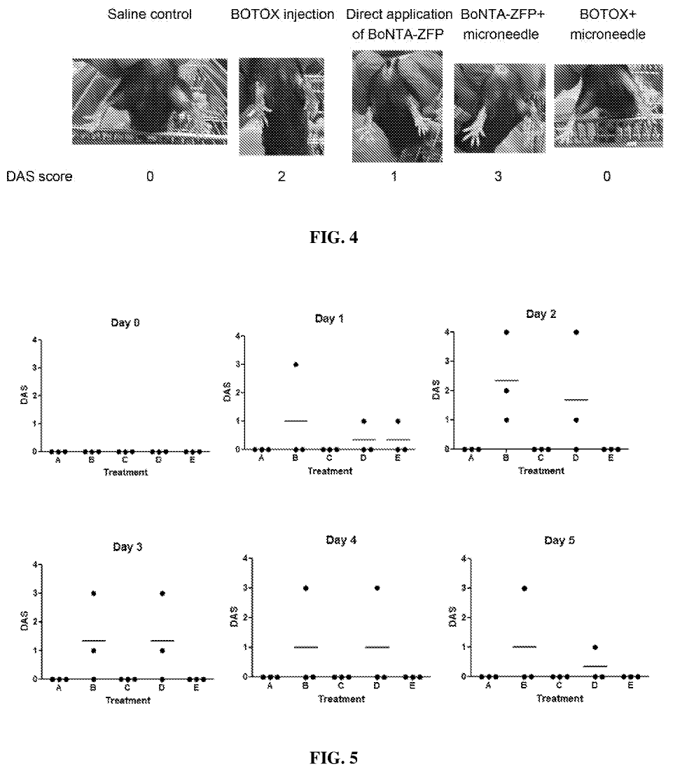

Both injectable BOTOX and BONTA-ZFP with microneedle pre-treatment exhibited notable digit abduction (FIG. 4), with significantly higher scores than control group A (p<0.05; Student's t test). Direct application of BoNTA-ZFP resulted in minimal effect. The digit abduction score (DAS) was evaluated by three independent researchers in a blinded manner. The DAS reached peak value at Day 2 (FIG. 5). This example therefore shows that when applied on to mouse skin, cell-penetrating BoNTA-ZFP can cause muscle paralysis, indicative of in vivo activity of SNAP-25 cleavage.

### Example 3. Intramuscular Administration of CPP-BONT Fusions Increased Therapeutic Index

The intramuscular therapeutic index of marketed BoNTA products typically range from 5 to 15. This example shows that fusion of cell-penetrating peptides (CPPs) to the 150 kDa core proteins of BoNTA could improve its intramuscular therapeutic index in mice by more than 10-fold. In addition, these CPP-BONTA exhibited reduced immunogenicity and longer duration in mice when administrated repeatedly.

### Plasmid Construction

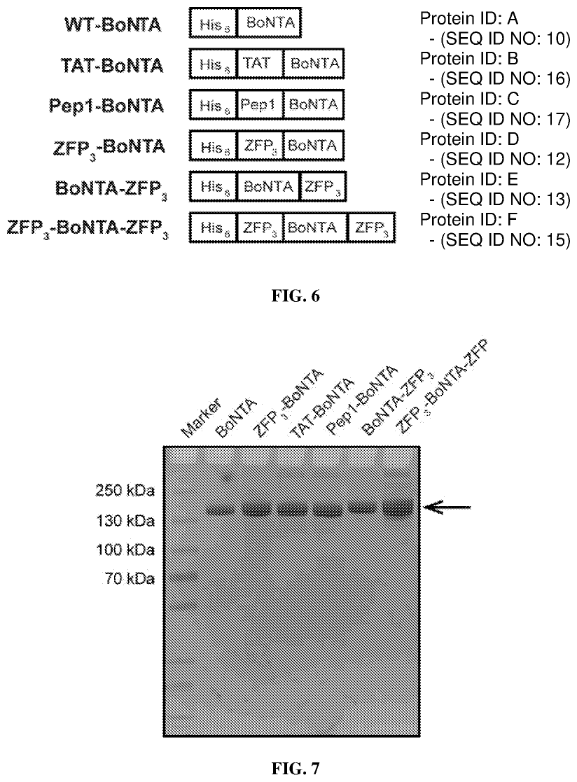

BONTA gene was synthesized by GENEWIZ Inc. (Nanjing, Jiangsu, China) and codon optimized for expression in Spodoptera frugiperda Sf9 cells or Trichoplusia ni Hi-5 cells. His₆ tag, FLAG tag, TEV cleavage site and GS linker were added into the fusion genes as indicated. The recombinant BoNTA and CPP-BONTA genes were cloned into the XbaI and HindIII sites of pFastBac1 vector. All the plasmid constructs were verified by Sanger sequencing and referred to as pFastBac-CPP-BoNTA thereafter. The tested fusion proteins are illustrated in FIG. 6, with sequences provided in Table 4.

### Production of Baculovirus

CPP-BONTA coding sequence was transferred from pFastBac to bacmid by transposition in DH10Bac Escherichia coli according to manufacturer's instructions of the Bac-to-Bac baculovirus expression system (Invitrogen, Carlsbad, California, USA). Recombinant bacmids were isolated and purified from E. coli cells using QIAGEN Large Construct Kit (QIAGEN, Germantown, Maryland, USA) according to manufacturer's protocol. Expression of CPP-BONTA in insect cells were performed using Bac-to-Bac baculovirus expression system (Invitrogen) by transfecting Sf9 cells with the recombinant bacmids. P1 baculoviral stock was collected and used to infect Sf9 cells to produce P2 virus. Briefly, 2 mL of P1 stock was supplemented into Sf9 cells at a density of 1.5×10⁶ cells per milliliter in 25 mL Sf-900 II medium (Gibco, Waltham, Massachusetts, USA). Sf9 cells were cultured at 27° C. for 72 h with shaking at 110 RPM in a fully humidified incubator. P2 viral stock was collected by centrifugation at 1000 g for 15 min to remove cells or cell debris. To generate high-titer P3 baculoviral stock, 1.5 mL P2 stock was added to Sf9 cells in 100 mL medium (1.5%, v/v) as described above. The transduced cells were cultured as above and P3 viral stock was collected at 72 h post infection.

### Protein Expression and Purification

To express CPP-BONTA, P3 viral stock was used to infect Hi-5 cells at a cell density of approximately 1.8˜ 2.0×10⁶ cells per milliliter in ESf 921 Insect Cell Culture Medium (Expression Systems, Davis, California, USA) with 1:100 dilution (v/v). The culture was incubated at 27° C. for 48 h and then the cells were harvested by centrifugation at 1000 g for 15 min.

Collected cells were lysed by sonication at 4° C. in a binding buffer containing 20 mM MOPS, 2 M NaCl. 10% glycerol, 10 μM ZnCl₂, pH 7.0 or pH 7.9 depending on the isoelectric point (μl) of the protein constructs. The cell lysate was centrifuged at 16,000 rpm for 30 min at 4 ºC. Cell supernatant was loaded onto a Ni²⁺-nitrilotriacetic acid (Ni-NTA) Sepharose affinity resin (QIAGEN) under native conditions. The resin was washed with 50× resin volumes of binding buffer and 6 resin volumes of wash buffer containing 20 mM MOPS, 100 mM NaCl, 10% glycerol. 10 μM ZnCl₂, pH 7.0 or pH 7.9. The bound proteins were eluted using an elution buffer containing 20 mM MOPS, 100 mM NaCl, 10% glycerol, 10 μM ZnCl₂, pH 7.0 or pH 7.9, and imidazole of gradient concentrations of 20 mM, 40 mM and 300 mM. Collected elution fractions were concentrated using spin concentrator with 30 kDa molecular weight cut off (MWCO) (Millipore, Burlington, Massachusetts, USA). The proteins were further purified using fast protein liquid chromatography with Superdex 200 Increase 10/300 GL column (GE Healthcare, Shanghai, China). The proteins were harvest and stored in storage buffer containing 20 mM MOPS, pH 7.0 or pH 7.9, 100 mM NaCl, 10% glycerol, at −80° C.

### Mass Spectrometry Analyses of CPP-BONTA

Samples were desalted using C18 ZipTips (Millipore) and eluted with 50% acetonitrile and 0.1% formic acid. Thereafter, samples were mixed with alpha-cyano-4-hydroxycinnamic acid (Agilent Technologies, Santa Clara, California, USA). Analyses were performed on a Bruker Autoflex MALDI-TOF mass spectrometer (Bruker, Billerica, Massachusetts, USA) in positive ion reflectron mode using standard operating conditions. Q-TOF Premier mass spectrometer (Waters, Milford, Massachusetts) equipped with a Waters nano-ESI source that is coupled directly to a Nano-Acquity UPLC system (Waters) with a 100 μm×15 cm reverse phase column (BEH C18, Waters) was used for all LC-MS/MS analyses. Mascot software (version 2.2.1. Matrix Science, London, UK) was used for database searching and spectral interpretation.

### In Vitro Peptide Cleavage Assay

The in vitro peptide cleavage assay was carried out by fluorescence resonance energy transfer (FRET). The peptide substrate contains sequence that is derived from the native BONTA substrate, SNAP-25. In the present example, we synthesized a substrate peptide with the sequence FITC-Thr-(D-Arg)-Ile-Asp-Gln-Ala-Asn-Gln-Arg-Ala-Thr-Lys-(DABCYL)-Nle-NH₂ (GL Biochem Corporation, Shanghai, China, SEQ ID NO: 135). In this peptide, the N-terminal fluorophore is fluorescein isothiocyanate (FITC) and C-terminal quencher is 4-((4-(dimethylamino) phenyl)azo) benzoic acid (DABCYL). Upon cleavage of the peptide, the fluorophore FITC will be released and the activated fluorescence signal can be measured spectroscopically. The synthesis procedure and characterization of the FRET peptide can be found in supplementary information. The cleavage reaction contains 20 mM HEPES, pH 7.4, 0.05% Tween 20, 100 nM recombinant CPP-BONTA and 10 μM SNAPtide substrate and was incubated at 37° C. for 40 min. The fluorescence was measured by a plate reader with an excitation wavelength of 490 nm and an emission wavelength of 523 nm.

### Cell Culture

Mouse neuroblastoma N2a cells were maintained in DMEM (Gbico) supplemented with 10% FBS (Gbico), 1% non-essential amino acids (Gbico) and 100 U ml⁻¹ penicillin/streptomycin (Gbico) at 37° C. in fully humidified atmosphere with 5% CO₂.

### Experimental Animals

All experiments were conducted in accordance with the guidelines of the American Association for the Accreditation of Laboratory Animal Care (AAALAC). All animal experimentation was conducted in accordance with the regulations of Animal Care and Use Committee, Shanghai Model Organisms Center, Inc. Eight week old, C57BL/6J female mice (17 to 22 g, Shanghai Model Organisms Center, Shanghai, China) were housed in a barrier facility and were maintained on a 12-h light/dark cycle (7 AM to 7 PM) with ad libitum access to food and water.

### Injection Procedure

Each mouse received intramuscular injection of CPP-BONTA or vehicle into the head of the right gastrocnemius muscle. Injections were made in a fixed volume of 5 μL using a 30 gauge needle attached to a sterile 250 μL Hamilton syringe. For each experiment, eight to ten mice were injected per dose. The experiments were performed with four to six biological replicates.

### Immunofluorescence Staining

Mouse neuroblastoma N2a cells were seeded on coverslips in culture dishes and grown to a confluency of 70% to 80%. Cells were then fixed using 4% paraformaldehyde (BBI Life Sciences Corporation, Shanghai, China) and permeabilized with phosphate buffered saline (PBS) containing 0.1% Triton X-100 for 10 min. Cells were blocked using blocking solution containing PBS supplemented with 3% bovine serum albumin (BSA) (Solarbio Life Sciences, Beijing, China) and incubated overnight with goat anti-FLAG (Novus Biologicals, Littleton, Colorado, USA) and rabbit anti-SV2A antibodies (Novus) at 1 to 200 dilution in PBS supplemented with 0.2% BSA. Cells were then washed with PBS supplemented with 0.2% BSA and incubated with Alexa568-conjugated donkey anti-rabbit IgG (Invitrogen) and Alexa488-conjugated donkey anti-goat IgG (Invitrogen) secondary antibodies.

The treated gastrocnemius muscles were sectioned, immediately fixed with 4% paraformaldehyde and dehydrated overnight in 30% sucrose. The tissue blocks were then dried on paper towel and placed on tissue molds that were sequentially filled with 100% optimal cutting temperature compound (OCT) over a total period of 4 h at −80ºC. OCT-embedded gastrocnemius muscles were serially frozen-sectioned at 10 μm interval along the horizontal direction. Section slices were blocked using blocking solution containing PBS and 5% FBS (Solarbio), then incubated with anti-FLAG antibody (Novus), washed with PBS and incubated with Alexa568-conjugated donkey anti-goat IgG antibody (A11057, Invitrogen).

Antibody-labeled cells and tissue sections were stained with Hoechst 33342 (Invitrogen) for nucleus visualization. Images were obtained using LSM710 laser scanning confocal microscopy (Carl Zeiss Microscopy GmbH, Jena, Germany) and TissueFAXS (TissueGnostics, Vienna, Austria) fluorescence imaging system. For confocal microscopy, the excitation/emission filters for red and green channels are 410 nm/507 nm and 493 nm/598 nm respectively. The fluorescence intensity in each cell was measured by ZEN 2011 imaging software (Zeiss). For the TissueFAXS, the whole section slices were scanned and fluorescence intensity was calculated based on nucleus staining with Hoechst 33342 (Invitrogen) using TissueQuest software (TissueGnostics).

### Digit Abduction Score (DAS) Assay

The mouse DAS assay was used to determine the pharmacologic activity of BONTA preparations by measuring the muscle weakening effectiveness. In the DAS assay, mice were briefly suspended by their tails to elicit a characteristic startle response in which the mice extended their hind-limbs and abducted their hind digits. Following BoNTA injection, the degrees of digit abduction were scored on a five-point scale by two separate observer, with greater scores indicating more muscle-weakening effects. The peak DAS response at each dose, which was typically observed on Day 2 or 3 post injection, was fit into linear or logarithmic regression equations for calculations of the half effective dose by intramuscular injection (IMED₅₀). The IMED₅₀ value was defined as the dose at which half of the mice produced a DAS value of 2.

### Determination of Systemic Effects and Therapeutic Index by Intramuscular Injection

The half lethal dose by intramuscular injection (IMLD₅₀) was defined as the dose at which 50% of the mice died following treatment. The end point of monitoring was set at day 5, after which no further death was found. This lethality reflects the systemic effects of BONTA considering neurotoxin escape from the muscle and its circulation through the whole body. The intramuscular therapeutic index, or margin of safety, of each sample was defined as the ratio between IMLD₅₀ and DAS IMED₅₀ values that were obtained from the same experiment.

### Gait Dynamic Assay

All gait dynamic assays were performed using the DigiGait imaging system along with Walk Analysator analysis software (Mobile Datum, Inc.; Shanghai, China). Briefly, mice were placed on the glass plate of Walk Analysator in a dark room and allowed for volunteer walk. The beam from a fluorescent lamp was focused on the glass plate and the reflection was set to horizontal direction. With forces during paw touches, the beam was reflected downwards. The images of paw print were captured by a digital camera (The Imaging Source Inc., Germany) at a rate of 120 frames/s and stored as audio video interleaved (AVI) files for subsequent analyses. For each paw, an average of 10 sequential strides were collected, which was validated in our experiments to be sufficient for analyzing the walking behavior of mice. Prior to examination, animals were habituated to explore the glass chamber three times a day.

### Treadmill Test

The treadmill test was performed on a motorized rodent treadmill machinery (Mobile Datum, Inc.; Shanghai, China) equipped with gradient inclination and an electric grid at the rear of the treadmill. Mice were habituated to treadmill one day before examination. For each test, a warm-up walking was assigned with a speed of 5 m min⁻¹ and no inclination. Since the start of the test, the speed of treadmill was increased every 5 min by 3 m min⁻¹ and the inclination was increased by 3° with a maximum grade of 12°. The end point of each run was defined as the mice staying at the last one third of the treadmill for more than 10 s or their repeated contact with the electric grid.

### Enzyme Linked Immunosorbent Assay

Repeated injection of CPP-BONTA or vehicle was performed after mice recovered from muscle paralysis. Blood samples were obtained from mouse orbit. Sera were collected and stored at −80° C. until use. ELISA plated were coated with antigens at 10 ng/ml in coating buffer at 4° ° C. overnight, washed for three times with 200 mL of 0.05% Tween in PBS (PBST) and blocked with PBS buffer supplemented with 5% BSA for 1 h. Sera (100 μL) with 1:10 dilution were added to each well of the microtiter plates and incubated for 1 h at room temperature. The plates washed and then incubated with HRP-conjugated goat anti-mouse IgG (R&D, HAF007) at 1:1000 dilution for 30 min at room temperature. The plates were washed for three times and the optical density at 405 nm (OD₄₅₀) was measured. BONTA and coating buffer were included as positive and negative controls respectively.

### Results

This example designed recombinant BoNTA proteins fused to various types of CPPS, including ZFPs, Pep1 and TAT (FIG. 6, Table 4). As illustrated (not showing signal peptides and other non-critical domains), compared to the wildtype (WT-BONTA) control, Protein ID: A (SEQ ID NO: 10), TAT-BONTA (Protein ID: B; SEQ ID NO: 16) includes a TAT peptide at the N-terminus of the BoNTA protein. Likewise, Pep1-BONTA (Protein ID: C; SEQ ID NO: 17) uses a Pep1 as the CPP instead, and ZFP₃-BONTA (Protein ID: D; SEQ ID NO: 12) includes three ZFP sequences. In BoNTA-ZFP₃ (Protein ID: E; SEQ ID NO: 13), the ZFP sequences are placed at the C-terminal side of the BoNTA sequence. Also prepared was ZFP₃-BONTA-ZFP₃ (Protein ID: F; SEQ ID NO: 15), in which ZFP₃ were inserted at both the N- and C-terminal sides of the BoNTA.

Wild-type (WT) and CPP-fused BONTA (CPP-BoNTA) proteins were expressed and purified with high homogeneity from insect cells using baculovirus expression system (FIG. 7). The whole molecular weight and N-terminal sequences of CPP-BONTA proteins were confirmed by mass spectrometry (MS) analyses.

Unexpectedly, these insect cell-produced BONTA proteins remained as intact peptide chains, rather than cleaved LC and HC.

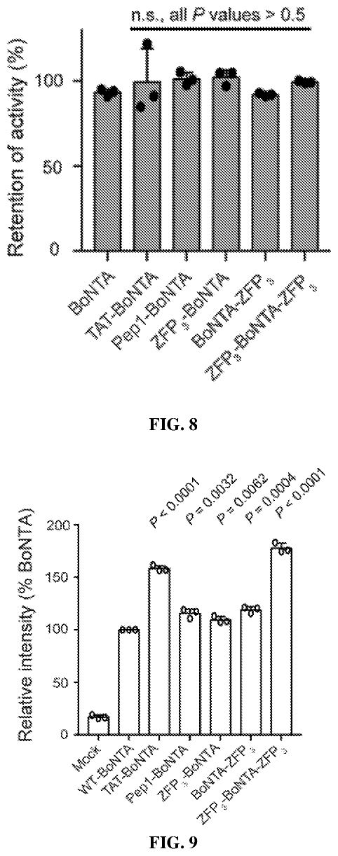

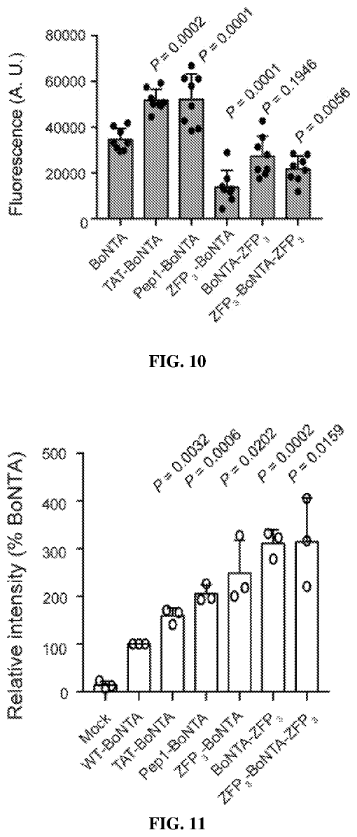

Next this example characterized the cleavage and cell-penetrating activities of CPP-BONTA. CPP fusion affected the peptide cleavage activity of BONTA by different manners and degrees, as determined using a fluorescence resonance energy transfer (FRET) peptide reporter. Both WT-BONTA and CPP-BONTA proteins had similar stability and could retain the majority of cleavage activities after incubation at 4° C. for one month (FIG. 8). Different CPPs enhanced cellular uptake of BoNTA by varying degrees, with TAT and bipartite ZFP fusion exhibiting highest improvement (FIG. 9). Importantly, internalized CPP-BONTA was found to co-localize with BoNTA receptor SV2. It appeared that the effects of CPP fusion on the cleavage and internalization activities of BONTA are unrelated (FIGS. 9 and 10). Consistently, CPP fusion enhanced the in vivo cellular uptake of intramuscularly injected BONTA (FIG. 11) in gastrocnemius muscles.

This example next characterized the intramuscular toxicity and potency of CPP-BONTA in mice. Home-purified BoNTA core protein (WT-BONTA) exhibited different pharmacological properties in comparison with marketed Botox (OnabotulinumtoxinA). This discrepancy may result from the distinct production procedures or chemical structures. It was found that all BONTA constructs with ZFP fusion showed lower toxicity (higher IMLD₅₀) than WT-BONTA with bipartite ZFP fusion (ZFP₃-BONTA-ZFP₃) displaying greatest improvement. Following conventional standard to define BoNTA potency using systemic lethality, we defined one active unit of BoNTA as the amount of proteins that result in 50% death via intramuscular injection. The in vivo efficacies of CPP-BONTA, as determined by digit abduction score (DAS) assay, were dose- and time-dependent with the peak effects observed typically at day 2 after treatment. We thus used DAS values at day 2 to determine the in vivo potency of WT-BONTA and CPP-BONTA. The IMED₅₀ of BONTA was defined as the amount of proteins that lead to half of the mice exhibiting a minimum DAS value of 2. It was found that all CPP-BONTA proteins have higher potency than WT-BONTA (lower IMED₅₀ values). Most importantly, compared with Botox or WT-BONTA, CPP-BONTA all showed increased therapeutic index, as defined by the difference between IMLD₅₀ and IMED₅₀, with up to 10-fold improvement observed with ZFP₃-BONTA-ZFP₃ (Table 5).

TABLE 5
Therapeutic Index of the Proteins

| | | | Therapeutic Index |
|---|---|---|---|
| | IMLD₅₀ | IMED₅₀ | IMLD₅₀ (ng/kg)/ |
| Samples | (ng/kg) | (ng/kg) | IMED₅₀ (ng/kg) |
| Botox | 3.41 | 0.12 | 28 |
| WT-BoNTA | 79.85 | 2.20 | 36 |
| TAT-BoNTA | 26.48 | 0.22 | 120 |
| Pep1-BoNTA | 34.89 | 0.37 | 94 |
| ZFP₃-BoNTA | 98.97 | 0.70 | 142 |
| BoNTA-ZFP₃ | 168.61 | 0.89 | 189 |
| ZFP₃-BoNTA-ZFP₃ | 780.89 | 2.77 | 282 |

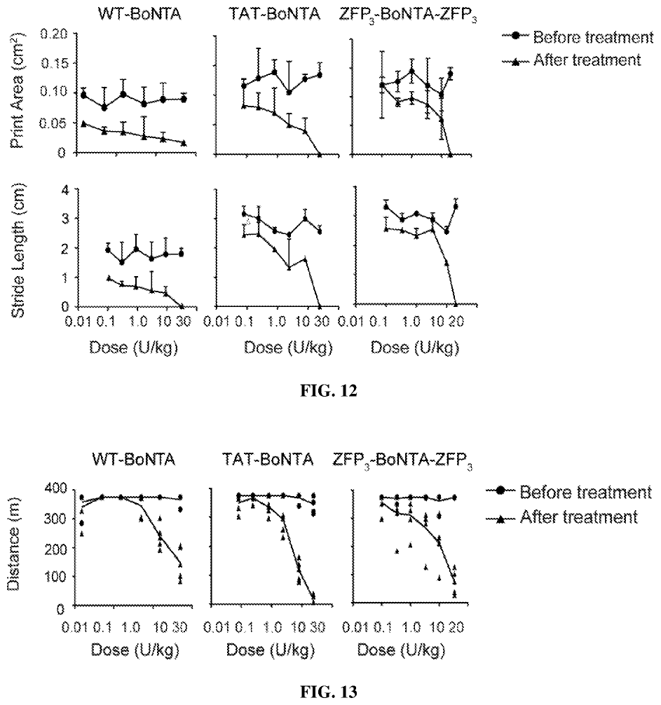

Because DAS is deemed as semi-quantitative analysis, we intended to characterize the muscle-paralyzing activities of top candidates of selected BONTA variants using fully quantitative gait and treadmill analyses. Taking the in vitro and in vivo performance into account, we chose WT-BONTA, TAT-BONTA and ZFP₃-BONTA-ZFP₃ for further investigation. Gait analysis showed that BoNTA injection could reduce the print areas and stride lengths of mice in a dose-dependent manner, indicative of muscle-weakening effects. It was fond that TAT-BONTA and ZFP₃-BONTA-ZFP₃ exhibited consistently higher potency than WT-BONTA (FIG. 12). Similarly, treadmill analysis revealed that intramuscular injection of BoNTA could reduce the running distance of mice and that CPP fusion enhanced the muscle-weakening effects of BONTA (FIG. 13). Importantly, gait and treadmill analyses were consistent with DAS in determination of IMED₅₀, supporting our conclusion that CPP fusion improved the therapeutic index of BONTA.

To explore the possible mechanism of action of CPP-mediated improvement of therapeutic index, we analyzed the immunogenicity of WT-BOTNA, TAT-BONTA and ZFP₃-BONTA-ZFP₃ at their minimum dosages of inducing maximum DAS values. It was found that during repeated dosing CPP-BONTA proteins exhibited lower titers of neutralizing antibodies compared with WT-BONTA (FIG. 14), suggesting that CPP fusion could reduce the immunogenicity of BoNTA. Consistently, CPP-BONTA displayed longer duration than WT-BONTA after repeated dosing despite shorter duration at initial dosing (FIG. 15). Such a superior property of these peptides allows them to be administered less frequently (e.g., once every 2 months, once every 3 months, once every 4 months, once every 5 months, or once every 6 months) than other products, such as Botox.

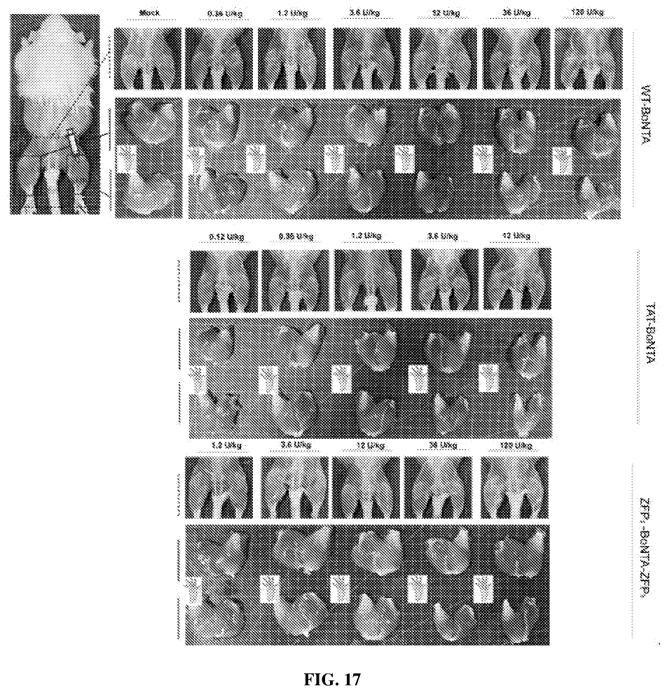

In addition, the physiological effects of the CPP fusions were examined. As shown in FIG. 16 (on gastrocnemius muscle hypertrophy in C57BL/6 mice) and FIG. 17 (on gastrocnemius muscle hypertrophy in SD rats), the CPP fusions improved the muscle shaping effect of wild-type BONTA.

This example therefore shows that CPP-BONT fusions, when intramuscularly administered, can improve the therapeutic index, immugenonicity and duration of purified BONTA proteins in mice. These proteins, produced in insect cells, surprisingly stayed as single-chains. Also surprisingly, even though single-chain BONT proteins were known as relatively inactive, these insect cell-produced BONT proteins were highly active in vitro and in vivo.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference

What is claimed is:

1. A method of delivering a botulinum toxin (BoNT) to a mammal, comprising intramuscularly administering a pharmaceutical composition comprising a polypeptide comprising a BoNT fused to a cell penetrating peptide (CPP), wherein the BoNT comprises a light chain and a heavy chain.

2. The method of claim 1, wherein the CPP is selected from the group consisting of a zinc finger peptide (ZFP, EKPYKCPECGKSFSASAALVAHQRTHTG, SEQ ID NO:1), TAT (GRKKRRQRRRPQ, SEQ ID NO:18), Pep-1 (KETWWETWWTEWSQPKKKRKV, SEQ ID NO:19), and those provided in Table 3.

3. The method of claim 1, wherein the CPP is fused to the N-terminus of the light chain of the BoNT.

4. The method of claim 1, wherein the CPP is fused to the C-terminus of the heavy chain of the BoNT.

5. The method of claim 1, comprising at least two CPPs, wherein at least one of the CPPs is fused to the N-terminus of the light chain, and another of the CPPs is fused to the C-terminus of the heavy chain.

6. (canceled)

7. The method of claim 1, wherein at least 50% of the BoNT in the composition are expressed from insect cells.

8. The method of claim 7, wherein the insect cells are Spodoptera frugiperda cells or Trichoplusia ni cells.

9. The method of claim 1, wherein the intramuscular administration is under a skin or a mucous membrane of an eye, or at an ear, nose, mouth, lip, urethral opening, anus, or tongue.

10. The method of claim 1, wherein the mammal is in need of treatment of facial wrinkle, dystonias, sparsticity, hemifacial spasm, hyperhidrosis, or hypersalivation.

11. The method of claim 1, wherein the mammal is in need of muscle shaping.

12. A pharmaceutical formulation comprising a pharmaceutically acceptable excipient and a polypeptide comprising a BoNT fused to a cell penetrating peptide (CPP), wherein the BoNT comprises a light chain and a heavy chain.

13. (canceled)

14. The pharmaceutical formulation of claim 12, which is an injectable solution.

15. The pharmaceutical formulation of claim 14, which is formulated for intramuscular injection.

16. The pharmaceutical formulation of claim 12, wherein the CPP is selected from the group consisting of a zinc finger peptide (ZFP, EKPYKCPECGKSFSASAALVAHQRTHTG, SEQ ID NO:1), TAT (GRKKRRQRRRPQ, SEQ ID NO:18), Pep-1 (KETWWETWWTEWSQPKKKRKV, SEQ ID NO:19), and those provided in Table 3.

17. The pharmaceutical formulation of claim 12, wherein the CPP is fused to the N-terminus of the light chain of the BONT.

18. The pharmaceutical formulation of claim 12, wherein the CPP is fused to the C-terminus of the heavy chain of the BoNT.

19. The pharmaceutical formulation of claim 12, comprising at least two CPPs, wherein at least one of the CPPs is fused to the N-terminus of the light chain, and another of the CPPs is fused to the C-terminus of the heavy chain.

20. (canceled)

21. The pharmaceutical formulation of claim 12, wherein at least 50% of the BoNT in the composition are expressed from insect cells.

22. The pharmaceutical formulation of claim 21, wherein the insect cells are Spodoptera frugiperda cells or Trichoplusia ni cells.

23. (canceled)

24. (canceled)

25. The method or pharmaceutical formulation of claim 12, wherein the BoNT is BONT A.
